(19) United States
(12) Patent Application Publication
(10) Pub. No.: US 2022/0008551 A1
(43) Pub. Date: Jan. 13, 2022

(54) POLYPEPTIDE CONJUGATES

(71) Applicant: ACADEMISCH ZIEKENHUIS LEIDEN (H.O.D.N. LUMC), Leiden (NL)

(72) Inventors: Dhr. Ferenc Alexander SCHEEREN, Leiden (NL); Dhr. Huib OVAA, Leiden (NL); Mw. Angela Fouad EL HEBIESHY, Leiden (NL)

(21) Appl. No.: 17/293,134

(22) PCT Filed: Nov. 18, 2019
(86) PCT No.: PCT/NL2019/050751

§ 371 Date: May 12, 2021

(30) Foreign Application Priority Data
Nov. 16, 2018 (NL) NL2022013

(51) Int. Cl. A61K 47/68; A61K 47/64

(52) U.S. Cl. CPC A61K 47/6835; A61K 47/641

(57) ABSTRACT

Disclosed herein is are conjugates that comprise a ubiquitin dimer or multimer, comprising a distal moiety conjugated to a proximal moiety. The distal moiety comprises a polypeptide comprising a distal ubiquitin at its C-terminus, said ubiquitin comprising at least one of the following mutations: K6X, K11X, K27X, K29X, K33X, K48X, K48X, K63X, or K63X, where X is selected from R, A or C. The proximal moiety comprises a polypeptide comprising either a proximal ubiquitin at its C-terminus or a proximal ubiquitin at its N-terminus; said ubiquitin comprising a blocked C-terminus. The distal moiety is conjugated to the proximal moiety via an amide bond from G76 of the distal ubiquitin to one of M1, K6, K11, K27, K29, K33, K48, or K63 of the proximal ubiquitin. Also provided are methods for the production of said conjugates, formulations comprising said conjugates and methods of using said conjugates.

This invention relates to polypeptide conjugates and methods for site-directed conjugation of polypeptides. The conjugates comprise ubiquitin linkages. The conjugates may comprise at least one UbiMab and/or at least one UbiFab, in which case the conjugates and methods may provide site directed antibody conjugates.

### BACKGROUND

Conjugated polypeptides generally and conjugated antibodies in particular are useful as tools in research and diagnostics and may also be used as therapeutics. Examples of conjugated antibodies include fluorescently labelled antibodies for imaging, bi-specific antibodies for dual targeting, and antibody-drug conjugates (ADRs) for selective delivery of cytotoxic agents.

An antibody-drug conjugate may provide a humanized or human monoclonal antibody conjugated with a highly cytotoxic small molecules (payloads) through chemical linkers. The antibody-drug conjugate combination enables selective delivery of a potent cytotoxic payload to target cancer cells, resulting in improved efficacy, reduced systemic toxicity, and preferable pharmacokinetics (PK)/pharmacodynamics (PD) and biodistribution compared to traditional chemotherapy.

Catumaxomab, a rat-mouse hybrid monoclonal antibody and one of the first trifunctional antibodies approved for therapeutic use, binds both CD3 on cytotoxic T cells and EpCAM on human adenocarcinomas. It is able to do this as it consists of one half (one heavy chain and one light chain) of an anti-EpCAM antibody; and one half of an anti-CD3 antibody, so that each molecule of catumaxomab can bind both EpCAM and CD3. In addition, the Fc-region can bind to an Fc receptor on accessory cells like other antibodies. While this is considered a trifunctional antibody, due to the presence of the Fc-region, the key specificities EpCAM and CD3 specificities of the two Fab regions of the hybrid antibody.

Available conjugated antibodies suffer from a number of limitations. For example, known conjugated antibodies may be heterogeneous, which may reduce their specificity and utility. Another limitation is the immunogenicity when conjugated antibodies are used therapeutically. There is therefore a need to provide improved conjugated antibodies.

### BRIEF SUMMARY OF THE DISCLOSURE

The invention provides conjugates comprising ubiquitin linkages and methods of making such conjugates, which provide specificity regarding the ubiquitin linkages formed. This may provide a high level of specificity and homogeneity in the resulting conjugates. In addition, as ubiquitin is one of the most abundant post-translational modifications in eukaryotes, the presence of ubiquitin linkages is likely to be non-immunogenic. Low immunogenicity is advantageous for any conjugates that may have clinical applications. In one aspect, the invention provides a conjugate that comprises a ubiquitin dimer or multimer, comprising a distal moiety conjugated to a proximal moiety. The distal moiety comprises a polypeptide comprising a distal ubiquitin at its C-terminus, said ubiquitin optionally comprising at least one of the following mutations: K6X, K11X, K27X, K29X, K33X, K48X, K63X, or K63X, where X is selected from R, A or C. The proximal moiety comprises a polypeptide comprising either a proximal ubiquitin at its C-terminus or a proximal ubiquitin at its N-terminus. The distal moiety is conjugated to the proximal moiety via an amide bond from G76 of the distal ubiquitin to one of M1, K6, K11, K27, K29, K33, K48, or K63 of the proximal ubiquitin. In an embodiment where the distal moiety is conjugated to the proximal moiety via an amide bond from G76 of the distal ubiquitin to M1 of the proximal ubiquitin; the distal moiety may not comprise any of the mutations K6X, K11X, K27X, K29X, K33X, K48X, K63X, or K63X, where X is selected from R, A or C. In an embodiment where the distal moiety is conjugated to the proximal moiety via an amide bond from G76 of the distal ubiquitin to one of K6, K11, K27, K29, K33, K48, or K63 of the proximal ubiquitin; the distal moiety is conjugated to the proximal moiety via an amide bond from G76 of the distal ubiquitin comprises a mutation K to X, where the mutation is at the K position in the distal ubiquitin corresponding to the K position of the proximal moiety involved in the amide bond.

Where the proximal moiety comprises a polypeptide comprising a proximal ubiquitin at its C-terminus, the proximal ubiquitin may comprise a blocked C-terminus. The proximal moiety may comprise a polypeptide comprising a proximal ubiquitin at its C-terminus, said ubiquitin (optionally) comprising a blocked C-terminus; wherein the distal moiety is conjugated to the proximal moiety via an amide bond from G76 of the distal ubiquitin to one of K6, K11, K27, K29, K33, K48, or K63 of the proximal ubiquitin.

A second aspect provides a ubiquitin multimer. The multimer comprises a most distal monomeric moiety conjugated to a most proximal monomeric moiety via n intermediate monomeric moieties, where n is an integer from 1 to 100. Each monomeric moiety comprises a polypeptide comprising a ubiquitin at its C-terminus. The most distal monomeric moiety conjugated to a first intermediate monomeric moiety via an amide bond from G76 of the most distal monomeric moiety's ubiquitin to one of K6, K11, K27, K29, K33, K48, or K63 of the first intermediate monomeric moiety's ubiquitin. The n^{th} intermediate monomeric moiety is conjugated to the most proximal monomeric moiety via an amide bond from G76 of the n^{th} intermediate monomeric moiety's ubiquitin to one of K6, K11, K27, K29, K33, K48, or K63 of the most proximal monomeric moiety's ubiquitin. As will be appreciated, in any given ubiquitin to ubiquitin amide bond in such a ubiquitin multimer, the ubiquitin providing the G76 may be considered the immediately distal ubiquitin and its moiety the immediately distal moiety; while the ubiquitin providing the K6, K11, K27, K29, K33, K48, or K63 may be considered the immediately proximal ubiquitin and its moiety the immediately proximal moiety.

The ubiquitin multimers of this aspect of the invention typically have the multimer conjugated via a ubiquitin chain. For example, the immediately distal monomeric moiety may be conjugated to the x^{th} intermediate monomeric moiety via an amide bond from G76 of the immediately distal monomeric moiety's ubiquitin to one of K6, K11, K27, K29, K33, K48, or K63 of the x^{th} intermediate monomeric moiety's ubiquitin; and the x^{th} intermediate monomeric moiety is conjugated the immediately proximal monomeric moiety via an amide bond from G76 of the x^{th} intermediate monomeric moiety's ubiquitin to one of K6, K11, K27, K29, K33, K48, or K63 of the immediately proximal monomeric moiety's ubiquitin. The immediately distal monomeric moiety is the most distal moiety when the x^{th} intermediate monomeric moiety is the first intermediate monomeric moiety, or the immediately distal monomeric moiety is the (x-1)^{th} intermediate monomeric moiety when the x^{th} intermediate monomeric moiety is any intermediate monomeric moiety other than the first intermediate monomeric moiety. The immediately proximal monomeric moiety is the most proximal moiety when the x^{th} intermediate monomeric moiety is the n^{th} intermediate monomeric moiety, or the immediately proximal monomeric moiety is the (x+1)^{th} intermediate monomeric moiety when the x^{th} intermediate monomeric moiety is any intermediate monomeric moiety other than the n^{th} intermediate monomeric moiety

A third aspect of the invention provides a method for the production of a conjugate. The method comprises: (i) providing a solution comprising a distal moiety, a proximal moiety, a ubiquitin activating enzyme (E1), a ubiquitin-conjugating enzyme (E2) and optionally a ubiquitin-ligating enzyme (E3); and (ii) forming a first conjugate. The distal moiety comprises a distal ubiquitin at its C-terminus, the distal ubiquitin optionally comprising at least one of the following mutations: K6X, K11X, K27X, K29X, K33X, K48X, K63X, or K63X, where X is selected from R, A or C. The proximal moiety comprises a polypeptide comprising either a proximal ubiquitin at its C-terminus or a proximal ubiquitin at its N-terminus; said ubiquitin comprising a blocked C-terminus. The first conjugate is formed such that the distal moiety is conjugated to the proximal moiety via an amide bond from G76 of the distal ubiquitin to one of M1, K6, K11, K27, K29, K33, K48, or K63 of the proximal ubiquitin. In an embodiment, the solution comprises a ubiquitin-conjugating enzyme (E2) but does not comprise a ubiquitin-ligating enzyme (E3).

In an embodiment where the distal moiety is conjugated to the proximal moiety via an amide bond from G76 of the distal ubiquitin to M1 of the proximal ubiquitin; the distal moiety may not comprise any of the mutations K6X, K11X, K27X, K29X, K33X, K48X, K63X, or K63X, where X is selected from R, A or C. In an embodiment where the distal moiety is conjugated to the proximal moiety via an amide bond from G76 of the distal ubiquitin to one of K6, K11, K27, K29, K33, K48, or K63 of the proximal ubiquitin; the distal moiety is conjugated to the proximal moiety via an amide bond from G76 of the distal ubiquitin comprises a mutation K to X, where the mutation is at the K position in the distal ubiquitin corresponding to the K position of the proximal moiety involved in the amide bond.

The proximal moiety may comprise a polypeptide comprising a proximal ubiquitin at its C-terminus, said ubiquitin comprising a blocked C-terminus; wherein the distal moiety is conjugated to the proximal moiety via an amide bond from G76 of the distal ubiquitin to one of K6, K11, K27, K29, K33, K48, or K63 of the proximal ubiquitin.

The solution may comprise a ubiquitin-conjugating enzyme (E2) and a ubiquitin-ligating enzyme (E3).

A fourth aspect provides a method for the production of a multimeric conjugate. The method comprises (i) providing a solution comprising a monomeric moiety, a ubiquitin activating enzyme (E1), a ubiquitin-conjugating enzyme (E2) and optionally a ubiquitin-ligating enzyme (E3); wherein the or each monomeric moiety comprises a ubiquitin having G-76 available for formation of an amide bond at its C-terminus. The method further comprises (ii) thereby forming the multimeric conjugate comprising at least three conjugated monomeric moieties, such that a first monomeric moiety is conjugated to a second monomeric moiety via an amide bond from G76 of the first monomeric moiety's ubiquitin to one of K6, K11, K27, K29, K33, K48, or K63 of the second monomeric moiety's ubiquitin; and the second monomeric moiety is conjugated to a third monomeric moiety via an amide bond from G76 of the second monomeric moiety's ubiquitin to one of K6, K11, K27, K29, K33, K48, or K63 of the third monomeric moiety's ubiquitin. The multimer produced may comprise 3, 4, 5, 6, 7, 8, 9, 10 or more monomeric moieties. When the multimer comprises 4 or more monomeric moieties, step (ii) may further comprise the third monomeric moiety is conjugated to a fourth monomeric moiety via an amide bond from G76 of the third monomeric moiety's ubiquitin to one of K6, K11, K27, K29, K33, K48, or K63 of the fourth monomeric moiety's ubiquitin. When the multimer comprises 5 or more monomeric moieties, step (ii) may further comprise the fourth monomeric moiety is conjugated to a fifth monomeric moiety via an amide bond from G76 of the fourth monomeric moiety's ubiquitin to one of K6, K11, K27, K29, K33, K48, or K63 of the fifth monomeric moiety's ubiquitin. When the multimer comprises 6 or more monomeric moieties, step (ii) may further comprise the fifth monomeric moiety is conjugated to a sixth monomeric moiety via an amide bond from G76 of the fifth monomeric moiety's ubiquitin to one of K6, K11, K27, K29, K33, K48, or K63 of the sixth monomeric moiety's ubiquitin. When the multimer comprises 7 or more monomeric moieties, step (ii) may further comprise the sixth monomeric moiety is conjugated to a seventh monomeric moiety via an amide bond from G76 of the sixth monomeric moiety's ubiquitin to one of K6, K11, K27, K29, K33, K48, or K63 of the seventh monomeric moiety's ubiquitin. When the multimer comprises 8 or more monomeric moieties, step (ii) may further comprise the seventh monomeric moiety is conjugated to an eighth monomeric moiety via an amide bond from G76 of the seventh monomeric moiety's ubiquitin to one of K6, K11, K27, K29, K33, K48, or K63 of the eighth monomeric moiety's ubiquitin. When the multimer comprises 9 or more monomeric moieties, step (ii) may further comprise the eighth monomeric moiety is conjugated to a ninth monomeric moiety via an amide bond from G76 of the eighth monomeric moiety's ubiquitin to one of K6, K11, K27, K29, K33, K48, or K63 of the ninth monomeric moiety's ubiquitin. When the multimer comprises 10 or more monomeric moieties, step (ii) may further comprise the ninth monomeric moiety is conjugated to a tenth monomeric moiety via an amide bond from G76 of the ninth monomeric moiety's ubiquitin to one of K6, K11, K27, K29, K33, K48, or K63 of the tenth monomeric moiety's ubiquitin.

In an embodiment, the solution comprises a ubiquitin-conjugating enzyme (E2) but does not comprise a ubiquitin-ligating enzyme (E3).

A fifth aspect of the invention provides a conjugate obtainable by or obtained by the method of the third or fourth aspects. In an embodiment, the conjugate is obtainable by or obtained by a method of the disclosure.

A sixth aspect of the invention provides a formulation comprising a conjugate of the invention and optionally a pharmaceutically acceptable carrier.

A seventh aspect of the invention provides a conjugate of the invention, or a formulation of the invention, for use as a medicament. The conjugate may comprise at least one UbiFab or UbiMab.

An eighth aspect of the invention provides a conjugate of the invention, or a formulation of the invention, for use in the treatment of cancer, an autoimmune disease, Alzheimer's disease, or a genetic disorder. The conjugate may comprise at least one UbiFab or UbiMab.

A ninth aspect of the invention comprises a method for the treatment of cancer an autoimmune disease, Alzheimer's disease, or a genetic disorder in a patient, by administering an effective amount of a conjugate of the invention to the patent. The conjugate may comprise at least one UbiFab or UbiMab.

A tenth aspect of the invention provides a method of deactivating a conjugate of the invention, comprising contacting the conjugate with a deubiquitinating enzyme, such that a linkage between at least two of the moieties of the conjugate is cleaved. The method of deactivating the conjugate may comprise contacting the conjugate with a deubiquitinating enzyme, such that a linkage between two of the moieties of the conjugate is cleaved.

### BRIEF DESCRIPTION OF THE DRAWINGS

Embodiments of the invention are further described hereinafter with reference to the accompanying drawings, in which:

FIG. 1 illustrates a UbiFab, which comprises a Fab (polypeptide that includes a VH, a CH1, a VL, and a CL immunoglobulin domain) attached to a ubiquitin. In this example, the ubiquitin is attached to the CH.

FIG. 2 shows an IgG1 and its subdomains, as well as illustrating examples of conjugates comprising ubiquitin dimers and multimers. The illustrated examples conjugates include (a) a UbiFab dimer, (b) a UbiFab trimer, (c) a dimer comprising a UbiFab and a ubiquitin substituted with a probe, (d) a trimer comprising a UbiFab dimer and a ubiquitin substituted with a probe.

FIG. 3 provides an outline of an enzyme cascade involved in an exemplary synthesis of a UbiFab dimer.

FIG. 4 illustrates (a) the formation of an exemplary UbiFab dimer; and (b) how the exemplary dimer may be further conjugated with a ubiquitin substituted with a probe, forming a trimer comprising a UbiFab dimer and a ubiquitin substituted with a probe.

FIG. 5 illustrates four different approaches to conjugating a further ubiquitin to ubiquitin dimer conjugate. A) A post-proximal UbiFab is conjugated to a UbiFab dimer using a different ubiquitin linkage type as the ubiquitin linkage type used in the dimer. B) A post-proximal UbiFab is conjugated to a UbiFab dimer using the same ubiquitin linkage type as the ubiquitin linkage type used in the dimer. C) A pre-distal UbiFab is conjugated to the distal ubiquitin of a UbiFab dimer using a different ubiquitin linkage type as the ubiquitin linkage type used in the dimer. D) A pre-distal UbiFab is conjugated to the proximal ubiquitin of a UbiFab dimer using a different ubiquitin linkage type as the ubiquitin linkage type used in the dimer.

FIG. 6 illustrates the use of hybridoma gene editing to produce UbiFabs.

FIG. 7 provides coomassie staining and western blot analysis of UbiFabs obtained from hybridoma culturing media after 2 and 5 days in presence or absence of ubiquitin-propargylamine. After 5 days, DUBs released in the culturing media cleave the C-terminal UbiFab his-tag. Adding a selective DUB inhibitor prevents the loss of the his-tag.

FIG. 8 provides A) purification of proximal UbiFabs. Culturing media containing proximal UbiFabs are purified using his-tag affinity purification followed by protein G affinity purification. B) Purification of distal UbiFabs by first depleting the sample from UbiFabs with an uncleaved his-tag, by passing it through a his-tag affinity column followed by protein G affinity purification. C) SDS-PAGE and western blot analysis of purified UbiFabs with or without the addition of 3-mercaptoethanol (13-ME).

FIG. 9 illustrates the result obtained by flowcytometry analysis of mouse splenocytes, for a comparable CD3 positive population when stained with anti-CD3 monoclonal antibodies or anti-CD3 UbiFabs.

FIG. 10 demonstrates an example of multimerization of UbiFabs by ubiquitinating enzymes.

FIG. 11 illustrates a site specific conjugation of proximal UbiFabs to synthetic TMR-UbK48A. A) The availability of the C-terminus of TMR-UbK48A only while lysine 48 is only available on UbiFabs, allows the specific K48 ubiquitin chain formation between the C-terminus of TMR-UbK48A and K48 on UbiFabs. B) Purification of conjugated UbiFabs using protein G affinity purification. C) Flowcytometry analysis shows comparable staining of CD3 positive cells with TAMRA-anti CD3 UbiFabs compared to unconjugated UbiFabs.

FIG. 12 illustrates a bi-specific UbiFab formation. A) Site-directed conjugation of proximal and distal UbiFabs giving rise to bi-specific UbiFabs. B) purification of bi-specific UbiFabs by protein G affinity purification.

FIG. 13 demonstrates exposure of the C-terminus of UbiFabs, by cleaving a C-terminus His-tag with a DUB. The top spectrum indicates the deconvoluted mass spectrum for a UbiFabs comprising heavy chain/Ubiquitin/His-tag, while the bottom spectrum provides the deconvoluted mass spectrum for a UbiFabs comprising heavy chain/Ubiquitin after action of the DUBs.

FIG. 14 illustrates the multimerization of UbiFabs using the E2 UbcH7 and E3 NIeL for the assembly of both K6- and K48-linked ubiquitin chains. A UbiFab was used where all lysine residues and the C-terminus were available for conjugation. After 3 hours, coomassie staining showed bands of high molecular weight which increased in intensity upon further incubation in presence of additional E2 and E3 enzymes. These bands correspond to the formation of UbiFab multimers linked at K6 and/or K48.

FIG. 15 illustrates cleavage of UbiFabs using a DUB. The DUB OTUB1, a K48-specific DUB, was used for the cleavage of K48 linked di-ubifabs. After 30 minutes, the band corresponding to the di-ubifab disappears while the band corresponding to ubifab monomers increases in intensity. This indicates that UbiFabs can be readily cleaved using DUBs.

FIG. 16 provides confirmation of removal of the His-tag, exposing the C-terminal glycine of the proximal UbiFab of a heterodimer. A) Deconvoluted mass spectral data of sample at time 0. B) deconvoluted mass spectral data after 30 minutes of reaction with deubiquitinating enzyme UCHL3, demonstrating 100% cleavage of the His-tag.

FIG. 17 provides gel imaging data, demonstrating the formation of a trimer comprising UbiFab heterodimer conjugated with Rhodamine-Ubiquitin, with incorporation of the Rodamine dye confirmed by fluorescence.

FIG. 18 illustrates the formation of a trimer where a post-proximal UbiFab was reacted with the C-terminal glycine of the proximal UbiFab of the heterodimer of FIG. 16.

FIG. 19 provides thermal unfolding data for conjugated UbiFab dimers and fluorescent labelled UbiFab and unconjugated UbiFab monomers. The thermal stability is comparable for all tested species, demonstrating that ubiquiting conjugation does not compromise the protein stability.

FIG. 20 illustrates the site specific conjugation of UbiFab to UbiPeptides. A) Conjugation of anti-DEC205 distal UbiFab with proximal UbiSSP. B) Conjugation of anti-DEC205 distal ubifab with proximal UbiSLP.

FIG. 21 illustrates the formation of MHC-I Ubi-multimers. A) Illustrates the overall reaction. B) Is a coomassie stained gel, confirming the formation of MHC-I Ubi-multimers.

FIG. 22 illustrates the results when the MHC-I Ubi-multimers were fluorescently labelled with either Rho-Ub75 or Rho-UbK48A. The gel at the top of the figure is the commassie image, while the bottom image provides the fluorescent imaging results.

FIG. 23 provides flow cytometry data showing OT-1 CD8+T-cells stained with different dilutions of MHC-I H2Kb Ubi-multimers, confirming that the functionality of the Rhodamine labelled MHC-I multimers. The top trace is for a 1:5 dilution, the second from top trace a 1:10 dilution, the third from top trace a 1:20 dilution, the fourth from top trace a 1:40 dilution, the fifth from top trace a 1:80 dilution and the bottom trace provides a negative control.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Unless specifically excluded, embodiments in the above specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims)

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

“Percentage of sequence identity” is determined by comparing two optimally aligned sequences over a comparison window, where the portion of the polynucleotide sequence or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. The percentage of identity may be determined using NCBI Basic Local Alignment Search Tool (BLAST) (Altschul et al., J. Mol. Biol., (1990) 215, 403-10). BLAST is available from several sources, including the National Center for Biological Information (NCBI, National Library of Medicine, 8600 Rockville Pike, Bethesda, Md., 20894, USA) and on the Internet, for use in connection with the sequence analysis programs blastp, blastn, blastx, tblastn and tblastx. Additional information can be found at the NCBI web site (https://www.ncbi.nlm.nih.gov/).

The term “substantial identity” of amino acid sequences (and of polypeptides or proteins having these amino acid sequences) normally means sequence identity of at least 75% compared to a reference sequence as determined using a standard program; preferably BLAST using standard parameters. Preferred percent identity of amino acids can be any integer from 75% to 100%. More preferred embodiments include amino acid sequences that have at least 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” may share amino acid sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Polypeptides that are “substantially identical” to a reference sequence may also differ by specified point mutations, where the specified point mutation or point mutations may or may not represent a conservative substitution; such mutations include lysine to arginine, lysine to alanine and lysine to cysteine.

The term “ubiquitin” includes reference to a (functional) amino acid sequence that is substantially identical to SEQ ID NO: 1. Ubiquitin may have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1. The ubiquitin may comprise 1, 2, 3, 4, 5, 6, 7 or 8 (for example 1, 2, 3 or 4; e.g. 1 or 2) mutations selected from K6X, K11X, K27X, K29X, K33X, K48X, K48X, K63X, or K63X, where X is selected from R, A or C. The ubiquitin may comprise 1, 2, 3, 4, 5, 6, 7 or 8 conservative substitutions. A ubiquitin may be a translated ubiquitin, such as a ubiquitin that is produced by a cell (e.g. a CHO cell or a hybridoma cell), optionally as part of a fusion protein. A ubiquitin may be a synthetic ubiquitin, such as a ubiquitin prepared by total linear synthesis using solid phase peptide synthesis, e.g. as described in F. El Oualid, et al., Angew. Chem. Int. Ed. Engl., (2010), 49(52), 10149-53; or D. S. Hameed, et al., Bioconjug Chem., (2017), 28(3), 805-15. A synthetic ubiquitin may comprise 1, 2, 3, 4, 5, 6, 7 or 8 unnatural amino acid substitutions in addition to or instead of conservative substitutions. A synthetic ubiquitin may comprise a probe, for example a synthetic ubiquitin may be substituted with a probe.

The term “blocked C-terminus” includes reference to a ubiquitin that does not have a G76 available for formation of an amide bond. The G76 may not be available for formation of an amide bond for various reasons; for example, the G76 may be deleted or substituted with another amino acid, or the G76 may already have formed an amide bond (e.g. G76-Z, where Z is a sequence of one or more amino acids). The G76 may already have formed an amide bond, because the ubiquitin may be at the N-terminus or a larger polypeptide. Where the blocked C-terminus comprises G-76-Z, Z may be a sequence of 2-20 amino acids. Z may be a polyhistidine (His-tag), e.g. comprising at least 4 amino acids, such as a hexa histidine-tag. Z may not be a polypeptide comprising a ubiquitin. Where the blocked C-terminus comprises a G76 that has already formed an amide bond (e.g. G76-Z), the blocked C-terminus may be unblocked by the action a deubiquitinating enzyme, e.g. UCHL3.

The term “exposed C-terminus” includes reference to a ubiquitin that has a G76 available for formation of an amide bond.

The term “isolated” means a biological component (such as a nucleic acid molecule or protein) that has been substantially separated or purified away from other biological components in the cell of the organism in which the component naturally occurs, i.e., other chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Variants, fragments or fusion proteins: The disclosed proteins and polypeptides include variants, fragments, and fusions thereof.

The terms “antibody” or “antibodies” as used herein refer to molecules or active (multivalent) fragments of molecules that bind to known antigens, particularly to immunoglobulin molecules and to immunologically active portions of immunoglobulin molecules, i.e. molecules that contain two binding sites that immunospecifically binds to the target antigen (the Ig-like 1 domain of MuSK in this instance). The immunoglobulin according to the invention can be of any class (IgG, IgM, IgD, IgE, IgA and IgY) or subclass (e.g. IgG1, IgG2, IgG3, IgG4, IgA1 and IgA2) of immunoglobulin molecule and based on heavy chain sequences from any species. For example, the species may be, but not limited to dogs, cats, horses, cows, pigs, guinea pigs, mice, rats and the like. The species may be a primate (e.g. a non-human primate). In a preferred example, the species is a human.

The term “antibody” or “antibodies” include monoclonal, polyclonal, chimeric, single chain, bispecific, human and humanized antibodies as well as active multivalent fragments thereof. Examples of active multivalent fragments of molecules that bind to known antigens and are useful include F(ab′)₂, F(ab′)₃, diabodies, triabodies, scFv-Fc and di-scFv and minibodies, including the products of a Fab immunoglobulin expression library and epitope-binding multivalent fragments of any of the antibodies and multivalent fragments mentioned above. The present invention provides conjugates that may combine, via ubiquitin linkage, two or more multivalent fragments. This may provide bifunctional (bispecific) conjugates.

In a particular example, the antibody may be a monoclonal antibody (Mab). As used herein, the term “monoclonal antibody” refers to an antibody that is mass produced in the laboratory from a single clone and that recognizes only one antigen. Monoclonal antibodies may be generated by any appropriate technique known in the art (e.g. by production in HEK or insect cells, or by generation of B cell hybridomas). Examples of production systems for recombinant antibodies are set out in Schirrmann T., et al., Front Biosci. (2008), 13:4576-94. Review. Monoclonal antibodies may also be produced, for example by production in hybridoma cells, using methods disclosed in, e.g.: Köhler, G. & Milstein, C., Nature (1975) 256, 495-497; Cheong, T.-C., et al., Nat. Commun., (2016) 7, 10934; Pogson, M., et al., Nat. Commun., (2016), 7, 12535; or Mason, D. M. et al. High-throughput antibody engineering in mammalian cells by CRISPR/Cas9-mediated homology-directed mutagenesis. Nucleic Acids Res. (2018). doi:10.1093/nar/gky550.

The term “human antibody” is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice results in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. R. (1985) p. 77; and Boerner, P., et al., J. Immunol. 147 (1991) 86-95).

The terms “Fab”, “Fab region”, “Fab portion” or “Fab fragment” are understood to define a polypeptide that includes a VH, a CH1, a VL, and a CL immunoglobulin domain. Fab may refer to this region in isolation (e.g. as part of a UbiFab), or this region in the context of an antibody molecule, as well as a full length immunoglobulin or immunoglobulin fragment. Typically a Fab region contains an entire light chain of an antibody. A Fab region can be taken to define “an arm” of an immunoglobulin molecule. It contains the epitope-binding portion of that Ig. The Fab region of a naturally occurring immunoglobulin can be obtained as a proteolytic fragment by a papain-digestion. A fusion protein may be engineered to comprise a Fab region of a naturally occurring immunoglobulin molecule. A “F(ab′)2 portion” is the proteolytic fragment of a pepsin-digested immunoglobulin. A “Fab′ portion” is the product resulting from reducing the disulfide bonds of an F(ab′)2 portion. As used herein the terms “Fab”, “Fab region”, “Fab portion” or “Fab fragment” may further include a hinge region that defines the C-terminal end of the antibody arm (cf. above). This hinge region corresponds to the hinge region found C-terminally of the CH1 domain within a full length immunoglobulin at which the arms of the antibody molecule can be taken to define a Y. The term hinge region is used in the art because an immunoglobulin has some flexibility at this region.

The term “UbiFab” includes reference to a fusion protein comprising an antigen binding antibody fragment (Fab or F(ab′)₂) and a ubiquitin. The antigen binding antibody fragment may be a Fab. The ubiquitin may be located at the C-terminus of the fusion protein.

The term “UbiMab” includes reference to a fusion protein comprising a monoclonal antibody and a ubiquitin. The ubiquitin may be located at the C-terminus of the fusion protein. The monoclonal antibody may be Rituximab, Trastuzumab, Alemtuzumab, or Omalizumab.

The term “ubiquitin linkage” includes reference to two ubiquitins that are attached by an amide bond from the C-terminus of a first ubiquitin to a primary amine of a second ubiquitin. The first ubiquitin may be considered distal to the second ubiquitin. The second ubiquitin may be considered proximal to the first ubiquitin. The C-terminus of the first ubiquitin may be G76. The primary amine of the second ubiquitin may be selected from its N-terminus (e.g. M1), or the sidechain of one of K6, K11, K27, K29, K33, K48, or K63. The linkage may be performed by a ubiquitin-conjugating enzyme (E2); or the linkage may be performed by an E2 enzyme and a ubiquitin-ligating enzyme (E3). The specific linkage type formed (e.g. G76 to M1, G76 to K6, G76 to K11, G76 to K27, G76 to K29, G76 to K33, G76 to K48, or G76 to K63) is dependent on the E2 enzyme or E2/E3 enzyme combination used. Multiple moieties comprising ubiquitin may be attached to each other in this manner, providing ubiquitin comprising multimers. The use of ubiquitin linkages in accordance with the invention may provide a number of advantages. Ubiquitin may be non-immunogenic. Thus using ubiquitin linkage may avoid or reduce immunogenicity of the resulting conjugate, particularly in comparison to other methods for linking, e.g. Fabs or Mab. Ubiquitin linkages made in accordance with the methods disclosed herein may be made with a high degree of specificity, thereby reducing heterogeneity.

The term “deubiquitinating enzyme” (DUB) refers to a peptidase that cleaves the peptide bond between a ubiquitin and the moiety to which it is attached. A given ubiquitin linkage (e.g. G76 to M1, G76 to K6, G76 to K11, G76 to K27, G76 to K29, G76 to K33, G76 to K48, or G76 to K63) may be cleaved by an appropriate DUB. Where an active conjugate comprises a ubiquitin linkage, cleavage of said ubiquitin linkage (e.g. with an appropriate DUB) may inactivate the conjugate.

The term “probe” includes reference to a payload or a label. Where a moiety is substituted with a probe, the probe may be a payload. The probe may be a label.

The term “payload” includes reference to a cytotoxic agent that may be conjugated to an antigen targeting agent such as an antibody or antigen targeting portion thereof, e.g. a Mab or Fab. Exemplary payloads include actinomycin, all-trans retinoic acid, azacytidine, azathioprine, bleomycin, bortezomib, carboplatin, capecitabine, cisplatin chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, vinorelbine.

The term “label” includes reference to a chemical moiety or protein that is directly or indirectly detectable (e.g. due to its spectral properties, conformation or activity). The label can be directly detectable (e.g. dye or fluorophore) or indirectly detectable (e.g. hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyser; and fluorescent labels (fluorophores), where the output signal is generated by the excitation of a suitable molecular adduct and that can be visualized by excitation with light that is absorbed by the dye or can be measured with standard fluorometers or imaging systems. The label may be a chemiluminescent substance, where the output signal is generated by chemical modification of the signal compound; a metal-containing substance; or an enzyme, where there occurs an enzyme-dependent secondary generation of signal, such as the formation of a coloured product from a colourless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a colorimetric substrate (e.g., tetramethylbenzidine (TMB)) or a fluorogenic substrate such as Amplex Red reagent (Molecular Probes, Inc.) to detect the presence of HRP. Numerous labels are known by those of skill in the art and include, but are not limited to, particles, fluorophores, haptens, enzymes and their colorimetric, fluorogenic and chemiluminescent substrates and other labels. Preferred labels include fluorophores, fluorescent proteins, haptens, and enzymes.

The invention concerns amongst other things the treatment of a disease. The term “treatment”, and the therapies encompassed by this invention, include the following and combinations thereof: (1) hindering, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “inhibit” (and “inhibiting”) includes reference to delaying, stopping, reducing the incidence of, reducing the risk of and/or reducing the severity of an event, state, disorder or condition. Inhibiting an event, state, disorder or condition may therefore include delaying or stopping initiation and/or progression of such, and reducing the risk of such occurring.

### Conjugates

The invention provides conjugates as previously described. Conjugates of the invention comprise at least two moieties that are joined by a ubiquitin linkage.

In one embodiment, the invention provides a conjugate that comprises a ubiquitin dimer or multimer, comprising a distal moiety conjugated to a proximal moiety. The distal moiety comprises a polypeptide comprising a distal ubiquitin at its C-terminus, said ubiquitin optionally comprising at least one of the following mutations: K6X, K11X, K27X, K29X, K33X, K48X, or K63X, where X is selected from R, A or C. The proximal moiety comprises a polypeptide comprising a proximal ubiquitin at its C-terminus, or a proximal ubiquitin at its N-terminus. The distal moiety is conjugated to the proximal moiety via an amide bond from G76 of the distal ubiquitin to one of M1, K6, K11, K27, K29, K33, K48, or K63 of the proximal ubiquitin. Where the distal moiety is conjugated to the proximal moiety via an amide bond from G76 of the distal ubiquitin to one of K6, K11, K27, K29, K33, K48, or K63 of the proximal ubiquitin; the distal ubiquitin comprises a mutation at its corresponding lysine, e.g. as set out in Table 1.

Where the distal moiety is conjugated to the proximal moiety via an amide bond from G76 of the distal ubiquitin to M1 of the proximal ubiquitin; the distal moiety may not comprise any of the mutations K6X, K11X, K27X, K29X, K33X, K48X, K63X, or K63X, where X is selected from R, A or C. Where the distal moiety is conjugated to the proximal moiety via an amide bond from G76 of the distal ubiquitin to K6 of the proximal ubiquitin; the distal ubiquitin comprises the mutation K6X. Where the distal moiety is conjugated to the proximal moiety via an amide bond from G76 of the distal ubiquitin to K11 of the proximal ubiquitin; the distal ubiquitin comprises the mutation K11X. Where the distal moiety is conjugated to the proximal moiety via an amide bond from G76 of the distal ubiquitin to K27 of the proximal ubiquitin; the distal ubiquitin comprises the mutation K27X. Where the distal moiety is conjugated to the proximal moiety via an amide bond from G76 of the distal ubiquitin to K29 of the proximal ubiquitin; the distal ubiquitin comprises the mutation K29X. Where the distal moiety is conjugated to the proximal moiety via an amide bond from G76 of the distal ubiquitin to K33 of the proximal ubiquitin; the distal ubiquitin comprises the mutation K33X. Where the distal moiety is conjugated to the proximal moiety via an amide bond from G76 of the distal ubiquitin to K48 of the proximal ubiquitin; the distal ubiquitin comprises the mutation K48X. Where the distal moiety is conjugated to the proximal moiety via an amide bond from G76 of the distal ubiquitin to K63 of the proximal ubiquitin; the distal ubiquitin comprises the mutation K63X.

TABLE 1
Ubiquitin linkages and corresponding mutation

| Linkage via amide from distal | Corresponding distal ubiquitin |
|---|---|
| ubiquitin G76 to proximal | mutation (X is A, R, |
| ubiquitin residue | or C; e.g. R or C) |
| M1 | — |
| K6 | K6X |
| K11 | K11X |
| K27 | K27X |
| K29 | K29X, |
| K33 | K33X |
| K48 | K48X |
| K63 | K63X |

Where the proximal moiety comprises a polypeptide comprising a proximal ubiquitin at its C-terminus, the proximal ubiquitin may comprise a blocked C-terminus, for example because in methods disclosed herein this will prevent the proximal ubiquitin acting as a distal ubiquitin during the conjugation reaction. Thus having a proximal ubiquitin comprising a blocked C-terminus during a method for producing the conjugate as disclosed herein may ensure that only a distal moiety may be conjugated to a proximal moiety. This ensures that the intended conjugates are provided with a high degree of homogeneity. After the conjugate is formed, the proximal ubiquitin may have its C-terminal unblocked. For example, where the C-terminus is blocked by a sequence of one or more amino acid (G76-Z, where Z is one or more amino acids), the C-terminus may be unblocked by cleavage of the amino acids.

Each ubiquitin in the conjugate may be substantially identical to SEQ ID NO: 1. Appropriate (functional) ubiquitin amino acid sequences may have at least 80% sequence identity to SEQ ID NO: 1, i.e. they may have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1. Suitably, percent identity is calculated as the percentage of identity to the entire length of the reference sequence (e.g. SEQ ID NO:1). In other words, appropriate (functional) ubiquitin amino acid sequences may vary from the sequence shown in SEQ ID NO:1 by one or several (e.g. two, three, four, five etc) amino acids.

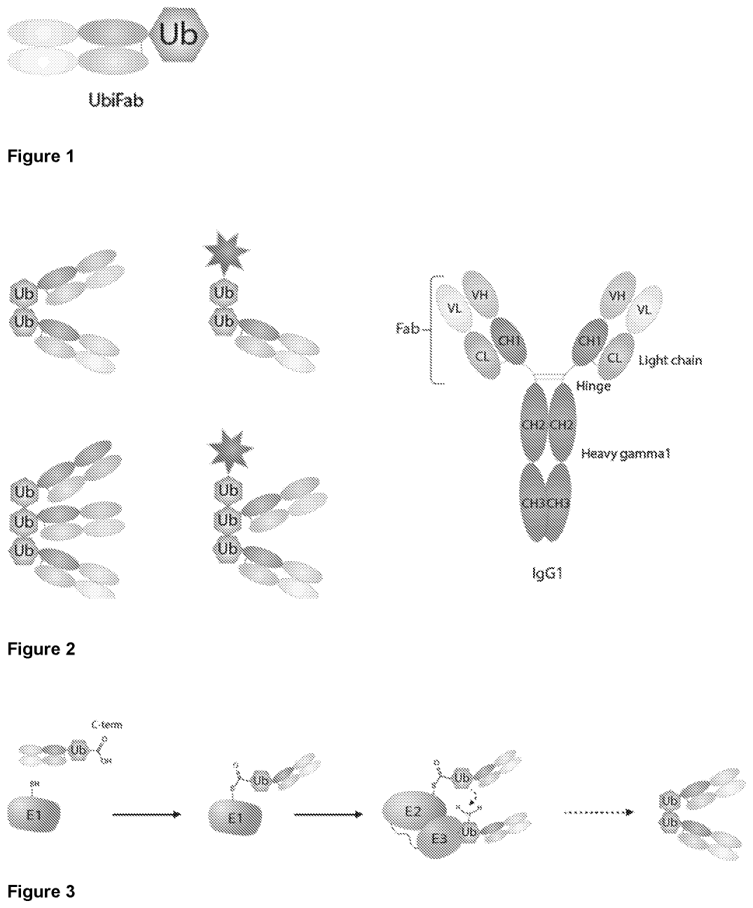

The conjugate may comprise a ubiquitin dimer. For example, the conjugate may comprise a UbiFab dimer as illustrated in FIG. 2, or a dimer comprising a UbiFab and a ubiquitin substituted with a probe as illustrated in FIG. 2. The conjugate may be a ubiquitin multimer; for example a ubiquitin trimer, tetramer or pentamer; e.g. a ubiquitin trimer. For example, the conjugate may comprise a UbiFab trimer as illustrated in FIG. 2, or a trimer comprising a UbiFab dimer and a ubiquitin substituted with a probe as illustrated in FIG. 2.

Where the conjugate is a ubiquitin trimer, the conjugate may comprise a proximal moiety and a distal moiety as defined herein, with a pre-distal moiety comprising a pre-distal ubiquitin conjugated to the distal moiety via an amide bond between G76 of the pre-distal ubiquitin to one of K6, K11, K27, K29, K33, K48, or K63 of the distal ubiquitin. Where the conjugate is a ubiquitin tetramer, the conjugate may comprise a proximal moiety, a distal moiety and a pre-distal moiety as defined herein; and further comprise a pre-pre-distal moiety comprising a pre-pre-distal ubiquitin conjugated to the pre-distal moiety via an amide bond between G76 of the pre-pre-distal ubiquitin to one of K6, K11, K27, K29, K33, K48, or K63 of the pre-distal ubiquitin. Where the conjugate is a ubiquitin pentamer or larger, the additional structure provided by each additional (pre)n-moiety (e.g. pre-pre-pre-distal moiety for a ubiquitin pentamer) can be defined in a likewise manner.

The distal moiety may comprise a fusion protein with ubiquitin at its C-terminus. The distal moiety may comprise a distal ubiquitin substituted with a probe. The proximal moiety may comprise a fusion protein with ubiquitin at its C-terminus. The proximal moiety may comprise a fusion protein with ubiquitin at its N-terminus. The proximal moiety may comprise a distal ubiquitin substituted with a probe. The distal moiety and the proximal moiety may both independently comprise a fusion protein with ubiquitin at its C-terminus; or one of the distal moiety and the proximal moiety may comprise a fusion protein and the other of the distal moiety and the proximal moiety may comprise a ubiquitin substituted with a probe. The pre-distal moiety may comprise a fusion protein with ubiquitin at its C-terminus; or the pre-distal moiety may comprise a probe. The pre-pre-distal moiety comprise a fusion protein with ubiquitin at its C-terminus; or the pre-distal moiety may comprise a probe.

The distal moiety may comprise a fusion protein comprising an active polypeptide or peptide and the distal ubiquitin at its C-terminus. The pre-distal moiety may comprise a fusion protein comprising an active polypeptide or peptide and the distal ubiquitin at its C-terminus. The pre-pre-distal moiety may comprise a fusion protein comprising an active polypeptide or peptide and the distal ubiquitin at its C-terminus. The proximal moiety may comprise a fusion protein comprising an active polypeptide or peptide and the proximal ubiquitin at its C-terminus.

An active polypeptide or peptide may be a biologically and/or pharmaceutically active polypeptide or peptide. For example, a biologically and/or pharmaceutically active polypeptide or peptide selected from an antigen binding antibody fragment (Fab); a monoclonal antibody (Mab); a major histocompatibility complex (MHC) polypeptide such as an MHC class I, an MHC class II, and MHC class III; an enzyme; a polypeptide or peptide drug or prodrug; or a polypeptide or peptide hapten.

The distal moiety may comprise a fusion protein comprising an antigen binding antibody fragment and the distal ubiquitin at its C-terminus (UbiFab), or a fusion protein comprising a monoclonal antibody and the distal ubiquitin at the C-terminus of the heavy chain or the light chain of the monoclonal antibody (UbiMab). The distal moiety may comprise a UbiFab. The distal moiety may comprise a UbiMab. The distal moiety may comprise a UbiMab where the distal ubiquitin is at the C-terminus of the heavy chain of the monoclonal antibody. The distal moiety may comprise a UbiMab where the distal ubiquitin is at the C-terminus of the light chain of the monoclonal antibody.

The proximal moiety may comprise a fusion protein comprising an antigen binding antibody fragment and the proximal ubiquitin at its C-terminus or N-terminus (UbiFab), or a fusion protein comprising a monoclonal antibody and the distal ubiquitin at the C-terminus (or N-terminus) of the heavy chain or the light chain of the monoclonal antibody (UbiMab). The proximal moiety may comprise a UbiFab where the proximal ubiquitin is at its C-terminus. The proximal moiety may comprise a UbiFab where the proximal ubiquitin is at its N-terminus. The proximal moiety may comprise a UbiMab. The proximal moiety may comprise a UbiMab where the proximal ubiquitin is at the C-terminus (or N-terminus) of the heavy chain of the monoclonal antibody. The proximal moiety may comprise a UbiMab where the proximal ubiquitin is at the C-terminus (or N-terminus) of the light chain of the monoclonal antibody.

The pre-distal moiety may comprise a fusion protein comprising an antigen binding antibody fragment and the pre-distal ubiquitin at its C-terminus (UbiFab), or a fusion protein comprising a monoclonal antibody and the pre-distal ubiquitin at the C-terminus of the heavy chain or the light chain of the monoclonal antibody (UbiMab). The pre-distal moiety may comprise a UbiFab. The pre-distal moiety may comprise a UbiMab. The pre-distal moiety may comprise a UbiMab where the pre-distal ubiquitin is at the C-terminus of the heavy chain of the monoclonal antibody. The pre-distal moiety may comprise a UbiMab where the pre-distal ubiquitin is at the C-terminus of the light chain of the monoclonal antibody.

The pre-pre-distal moiety may comprise a fusion protein comprising an antigen binding antibody fragment and the pre-pre-distal ubiquitin at its C-terminus (UbiFab), or a fusion protein comprising a monoclonal antibody and the pre-pre-distal ubiquitin at the C-terminus of the heavy chain or the light chain of the monoclonal antibody (UbiMab). The pre-pre-distal moiety may comprise a UbiFab. The pre-pre-distal moiety may comprise a UbiMab. The pre-pre-distal moiety may comprise a UbiMab where the pre-pre-distal ubiquitin is at the C-terminus of the heavy chain of the monoclonal antibody. The pre-pre-distal moiety may comprise a UbiMab where the pre-pre-distal ubiquitin is at the C-terminus of the light chain of the monoclonal antibody.

The distal moiety may be a UbiFab and the proximal moiety may be a UbiFab. The pre-distal moiety may comprise a pre-distal ubiquitin substituted with a probe, the distal moiety may comprise a UbiFab and the proximal moiety may comprise a UbiFab; or the pre-distal moiety may comprise a UbiFab, the distal moiety may comprise a UbiFab and the proximal moiety may comprise a proximal ubiquitin substituted with a probe. At least one of the distal ubiquitin and the proximal ubiquitin may be substituted with a probe. For example, the distal moiety may comprise a UbiMab and the proximal moiety may comprise a proximal ubiquitin substituted with a probe; or the distal moiety may comprise a distal ubiquitin substituted with a probe and the proximal moiety may comprise a UbiMab.

The distal ubiquitin may comprise at least one (e.g. one or two) of the following mutations: K6X, K11X, K27X, K29X, K33X, K48X, or K63X, where X is selected from R, A or C (e.g. R or C). For example, the distal ubiquitin may comprise at least one (e.g. one or two) of the following mutations: K6X, K11X, K29X, K33X, K48X, or K63X, where X is selected from R, A or C (e.g. R or C). For example, the distal ubiquitin may comprise K48X, where X is selected from R, A or C (e.g. R or C). The distal ubiquitin may comprise at least one (e.g. one or two) of the following mutations: K6R, K11R, K27R, K29R, K33R, K48R, K48C, K63R, or K63C. For example, the distal ubiquitin may comprise at least one mutation selected from K6R, K11R, K29R, K33R, K48R, K48C, K63R, or K63C; e.g. the distal ubiquitin may comprise K48R.

The distal moiety may be conjugated to the proximal moiety via an amide bond from G76 of the distal ubiquitin to one of M1, K6, K11, K27, K29, K33, K48, or K63 of the proximal ubiquitin. The distal moiety may be conjugated to the proximal moiety via an amide bond from G76 of the distal ubiquitin to one of K6, K11, K27, K29, K33, K48, or K63 of the proximal ubiquitin. The distal moiety may be conjugated to the proximal moiety via an amide bond from G76 of the distal ubiquitin to one of K6, K11, K29, K33, K48, or K63 of the proximal ubiquitin. For example, the distal moiety may be conjugated to the proximal moiety via an amide bond from G76 of the distal ubiquitin to one of K48 or K63 of the proximal ubiquitin; e.g. the distal moiety may be conjugated to the proximal moiety via an amide bond from G76 of the distal ubiquitin to K48 of the proximal ubiquitin.

The pre-distal ubiquitin may comprise at least one (e.g. one or two) of the following mutations: K6X, K11X, K27X, K29X, K33X, K48X, or K63X, where X is selected from R, A or C (e.g. R or C). For example, the pre-distal ubiquitin may comprise at least one (e.g. one or two) of the following mutations: K6X, K11X, K29X, K33X, K48X, or K63X, where X is selected from R, A or C (e.g. R or C). For example, the pre-distal ubiquitin may comprise K48X, where X is selected from R, A or C (e.g. R or C). The pre-distal ubiquitin may comprise at least one (e.g. one or two) of the following mutations: K6R, K11R, K27R, K29R, K33R, K48R, K48C, K63R, or K63C. For example, the pre-distal ubiquitin may comprise at least one mutation selected from K6R, K11R, K29R, K33R, K48R, K48C, K63R, or K63C; e.g. the pre-distal ubiquitin may comprise K48R.

The pre-distal moiety may be conjugated to the distal moiety via an amide bond from G76 of the pre-distal ubiquitin to one of K6, K11, K27, K29, K33, K48, or K63 of the distal ubiquitin. The pre-distal moiety may be conjugated to the distal moiety via an amide bond from G76 of the pre-distal ubiquitin to one of K6, K11, K29, K33, K48, or K63 of the distal ubiquitin. For example, the pre-distal moiety may be conjugated to the distal moiety via an amide bond from G76 of the pre-distal ubiquitin to one of K48 or K63 of the distal ubiquitin; e.g. the pre-distal moiety may be conjugated to the distal moiety via an amide bond from G76 of the pre-distal ubiquitin to K48 of the distal ubiquitin.

The pre-pre-distal ubiquitin may comprise at least one (e.g. one or two) of the following mutations: K6X, K11X, K27X, K29X, K33X, K48X, or K63X, where X is selected from R, A or C (e.g. R or C). For example, the pre-pre-distal ubiquitin may comprise at least one (e.g. one or two) of the following mutations: K6X, K11X, K29X, K33X, K48X, K48X, K63X, or K63X, where X is selected from R, A or C (e.g. R or C). For example, the pre-pre-distal ubiquitin may comprise K48X, where X is selected from R, A or C (e.g. R or C). The pre-pre-distal ubiquitin may comprise at least one (e.g. one or two) of the following mutations: K6R, K11R, K27R, K29R, K33R, K48R, K48C, K63R, or K63C. For example, the pre-pre-distal ubiquitin may comprise at least one mutation selected from K6R, K11R, K29R, K33R, K48R, K48C, K63R, or K63C; e.g. the pre-pre-distal ubiquitin may comprise K48R.

The pre-pre-distal moiety may be conjugated to the pre-distal moiety via an amide bond from G76 of the pre-pre-distal ubiquitin to one of K6, K11, K27, K29, K33, K48, or K63 of the pre-distal ubiquitin. The pre-pre-distal moiety may be conjugated to the pre-distal moiety via an amide bond from G76 of the pre-pre-distal ubiquitin to one of K6, K11, K29, K33, K48, or K63 of the pre-distal ubiquitin. For example, the pre-pre-distal moiety may be conjugated to the pre-distal moiety via an amide bond from G76 of the pre-pre-distal ubiquitin to one of K48 or K63 of the pre-distal ubiquitin; e.g. the pre-pre-distal moiety may be conjugated to the pre-distal moiety via an amide bond from G76 of the pre-pre-distal ubiquitin to K48 of the pre-distal ubiquitin.

A ubiquitin comprises a blocked C-terminus when it does not have a G76 available for formation of an amide bond. Thus an example of a blocked ubiquitin is the distal ubiquitin of a conjugate, where the distal ubiquitin comprises a ubiquitin linkage via its G76 to the proximal ubiquitin of the conjugate.

A proximal ubiquitin may comprise a blocked C-terminus. For example, the blocked C-terminus may comprise a deleted G76, or a G76-Z, where —Z is a sequence of one or more amino acids (e.g. 2-20 amino acids). While the exact length of the sequence Z is not critical (e.g. Z may be 1 to 100, or 2 to 100 or more amino acids), there does not appear to be an advantage in having a Z that is too long, so sequences with a modest length, such as Z is 2 to 20 amino acids, may be preferred. The blocked C-terminus may comprise a deleted G76. The blocked C-terminus may comprise a G76-Z. The −Z may be a polyhistidine (His-tag), e.g. comprising at least 4 amino acids, such as a hexa histidine-tag.

The distal moiety may be conjugated to the proximal moiety via an amide bond from G76 of the distal ubiquitin to one of M1, K6, K11, K27, K29, K33, K48, or K63 of the proximal ubiquitin. The distal moiety may be conjugated to the proximal moiety via an amide bond from G76 of the distal ubiquitin to one of K6, K11, K27, K29, K33, K48, or K63 of the proximal ubiquitin. The distal moiety may be conjugated to the proximal moiety via an amide bond from G76 of the distal ubiquitin to one of K6, K11, K29, K33, K48, or K63 of the proximal ubiquitin. For example, the distal moiety may be conjugated to the proximal moiety via an amide bond from G76 of the distal ubiquitin to one of K48 or K63 of the proximal ubiquitin; e.g. the distal moiety may be conjugated to the proximal moiety via an amide bond from G76 of the distal ubiquitin to K48 of the proximal ubiquitin.

The conjugate may further comprise a pre-distal moiety conjugated to the distal moiety. Said pre-distal moiety comprises a pre-distal ubiquitin at its C-terminus, said ubiquitin comprising at least one of the following mutations: K6X, K11X, K27X, K29X, K33X, K48X, or K63X, where X is selected from R, A or C. The pre-distal moiety is conjugated to the distal moiety via an amide bond from G76 of the pre-distal ubiquitin to one of K6, K11, K27, K29, K33, K48, or K63 of the distal ubiquitin.

The conjugate may further comprise a pre-pre-distal moiety conjugated to the pre-distal moiety. Said pre-pre-distal moiety comprises a pre-pre-distal ubiquitin at its C-terminus, said ubiquitin comprising at least one of the following mutations: K6X, K11X, K27X, K29X, K33X, K48X, or K63X, where X is selected from R, A or C. The pre-pre-distal moiety is conjugated to the pre-distal moiety via an amide bond from G76 of the pre-pre-distal ubiquitin to one of K6, K11, K27, K29, K33, K48, or K63 of the pre-distal ubiquitin.

A bi-functional conjugate may be provided by a conjugate comprising at least two ubiquitin comprising moieties, e.g. connected by ubiquitin linkages. In an exemplary bi-functional conjugate, the non-ubiquitin portion of the distal moiety and the non-ubiquitin moiety portion of the proximal moiety may differ, thereby providing a bi-functional conjugate. For example, where the distal moiety and proximal moiety both comprise UbiFabs (or UbiMabs) the Fab (or Mab) of each of the distal moiety and proximal moiety may have different specificities, thereby providing a bifunctional conjugate. For example, one of the distal moiety and proximal moiety could comprise a UbiFab or UbiMab, while the other comprises a ubiquitin substituted with a probe, thereby providing a bifunctional conjugate. A tri-functional conjugate may be provided by a conjugate comprising at least three ubiquitin comprising moieties, e.g. connected by ubiquitin linkages. In an exemplary tri-functional conjugate, the non-ubiquitin portion of the pre-distal moiety, the non-ubiquitin moiety portion of the distal moiety and the non-ubiquitin moiety portion of the proximal moiety may all differ, thereby providing a tri-functional conjugate. For example, two of said moieties may comprise UbiFabs (or UbiMabs) with different specificities, while the third said moiety may comprise a ubiquitin substituted with a probe. In a likewise manner, a tetra-functional conjugate may be provided by a conjugate comprising at least four ubiquitin comprising moieties (e.g. a pre-pre-distal moiety, pre-distal moiety, distal moiety and proximal moiety) e.g. connected by ubiquitin linkages; or a penta-functional monomer may be provided by a conjugate comprising at least five ubiquitin comprising moieties e.g. connected by ubiquitin linkages.

Where the conjugate is a ubiquitin trimer or higher order multimer, the conjugate may comprise monomeric moieties as defined herein, wherein the conjugate comprises the following structure: a first (most distal) monomeric moiety conjugated to a second monomeric moiety via an amide bond from G76 of the first monomeric moiety's ubiquitin to one of K6, K11, K27, K29, K33, K48, or K63 of the second monomeric moiety's ubiquitin; a second (intermediate) monomeric moiety is conjugated to a third monomeric moiety via an amide bond from G76 of the first monomeric moiety's ubiquitin to one of K6, K11, K27, K29, K33, K48, or K63 of the third monomeric moiety's ubiquitin. The final (most proximal) monomeric moiety (e.g. the third monomeric moiety in a ubiquitin trimer, or the fourth monomeric moiety in a ubiquitin tetramer) has the G76 of its ubiquitin available to form an amide bond. The conjugate may comprise a most distal monomeric moiety conjugated to a most proximal monomeric moiety via n intermediate monomeric moieties, where n is an integer from 1 to 100. For example, n may be at least 2, at least 3, at least 4, at least 5, or at least 6. For example n may be not more than 80, not more than 60, not more than 50, not more than 40, not more than 30, not more than 20 or not more than 10. For example, n may be an integer of from 1 to 30, e.g. of from 2 to 20. The or each of the monomeric moieties may have the features of a distal moiety as disclosed herein.

Each (relatively distal) monomeric moiety may be conjugated to the next (relatively proximal) moiety via an amide bond from G76 of the relatively distal monomeric moiety's ubiquitin to one of K6, K11, K27, K29, K33, K48, or K63 of the relatively proximal moiety's ubiquitin. The relatively distal moiety may be conjugated to the relatively proximal moiety via an amide bond from G76 of the relatively distal moiety's ubiquitin to one of K6, K11, K27, K29, K33, K48, or K63 of the relatively proximal moiety's ubiquitin. The relatively distal moiety may be conjugated to the relatively proximal moiety via an amide bond from G76 of the relatively distal moiety's distal ubiquitin to one of K6, K11, K29, K33, K48, or K63 of the relatively proximal moiety's ubiquitin. For example, the relatively distal moiety may be conjugated to the relatively proximal moiety via an amide bond from G76 of the relatively distal moiety's ubiquitin to one of K48 or K63 of the relatively proximal moiety's ubiquitin; e.g. the relatively distal moiety may be conjugated to the relatively proximal moiety via an amide bond from G76 of the relatively distal moiety's ubiquitin to K48 of the relatively proximal moiety's ubiquitin.

The or each of the monomeric moieties may comprise a fusion protein comprising an active polypeptide or peptide and a ubiquitin at its C-terminus. An active polypeptide or peptide may be a biologically and/or pharmaceutically active polypeptide or peptide. For example, a biologically and/or pharmaceutically active polypeptide or peptide selected from an I major histocompatibility complex (MHC) polypeptide such as an MHC class I, an MHC class II, and MHC class III (e,g. an MHC class I or an MHC class II); an enzyme; a polypeptide or peptide drug or prodrug; or a polypeptide or peptide hapten. The biologically and/or pharmaceutically active polypeptide or peptide may be a Fab. The biologically and/or pharmaceutically active polypeptide or peptide may be a Mab. The biologically and/or pharmaceutically active polypeptide or peptide may be an MHC.

The conjugate that comprises monomeric moieties may also comprise a ubiquitin comprising a probe. For example, one or more of the monomeric moieties may comprise a ubiquitin comprising a probe; e.g., the most distal monomeric moiety and/or most proximal monomeric moiety may comprise a ubiquitin substituted with a probe.

Each of the monomeric moieties may be the same. Each monomeric moiety may comprise an MHC (e.g. MHC class I, MHC class II, MHC class III) and a ubiquitin at its C-terminus, for example each monomeric moiety may comprise the same MHC (e.g. the same MHC class I, MHC class II, MHC class III) and a ubiquitin at its C-terminus.

Each of the monomeric moieties may be the same, other than the most distal monomeric moiety and/or most proximal monomeric moiety, either or both of which may comprise a ubiquitin substituted with a probe. For example, each monomeric moiety, other than the most distal monomeric moiety and/or most proximal monomeric moiety, may comprise an MHC (e.g. MHC class I, MHC class II, or MHC class III; such as an MHC class I, or MHC class II) and a ubiquitin at its C-terminus. For example, each monomeric moiety, other than the most distal monomeric moiety and/or most proximal monomeric moiety, may comprise the same MHC (e.g. the same MHC class I, MHC class II, or MHC class III; such as an MHC class I, or MHC class II) and a ubiquitin at its C-terminus. The most distal monomeric moiety may comprise a probe. The most proximal monomeric moiety may comprise a probe.

### Methods of Forming Conjugates

Conjugates of the disclosure may be made according to the methods provided herein. The methods provide a novel way to form site-directed conjugates (e.g. site directed antibody conjugates) using the non-immunogenic small protein ubiquitin. The ubiquitin machinery allows ubiquitin to cross-link either with another ubiquitin or with a different protein by forming a covalent bond between the C-terminal carboxy group of G76 of a ubiquitin and the primary amino group of the N-terminus or a lysine side chain of a target protein (S. Faggiano, et al., Analytical Biochemistry, (2016) 492, 82-90. The methods of the present invention involve novel manipulation of elements of the ubiquitin machinery to provide conjugates that are site-specifically joined through ubiquitin dimers or chains with specifically controlled ubiquitin linkage types.

In general terms, the processes of forming conjugates disclosed herein comprise an enzymatic cascade involving ubiquitin-activating (E1), ubiquitin-conjugating (E2) and often a ubiquitin-ligating (E3) enzymes, resulting in the covalent attachment of the C-terminal glycine residue of ubiquitin to the N-terminus or lysine residues of another ubiquitin. These processes are typically performed ex vivo. An example of this process, as used in the synthesis of conjugate comprising a UbiFab dimer, is illustrated in FIG. 3. As illustrated in FIG. 3, a distal UbiFab is recruited by an E1 enzyme, then transferred to an E2/E3 enzyme system. The E2/E3 enzyme system catalyzes the reaction between the C-terminus carboxyl group of G76 of the distal ubiquitin and a primary amino group of the N-terminus (M1) or a lysine side chain (K6, K11, K27, K29, K33, K48, or K63) of the proximal ubiquitin. While FIG. 3 illustrates use of an E2/E3 enzyme system, this is not always required, as some E2 enzymes can catalyse the reaction without an E3 enzyme; i.e. the method may comprise transfer of the distal UbiFab to an E2 enzyme and the E2 enzyme then catalysing the reaction between the C-terminus carboxyl group of G76 of the distal ubiquitin and a primary amino group of the N-terminus (M1) or a lysine side chain (K6, K11, K27, K29, K33, K48, or K63) of the proximal ubiquitin. As the skilled person will appreciate, the process of FIG. 3 is illustrated with UbiFab distal and proximal moieties, the process may be readily generalised to other types of distal moieties comprising a distal ubiquitin and proximal moieties comprising a proximal ubiquitin as disclosed herein. For example, either or both of the distal and proximal ubiquitin could comprise a UbiMab, or a ubiquitin (e.g. a synthetic ubiquitin) comprising a probe.

The methods disclosed herein typically provide site specificity of attachment, resulting in a high level of homogeneity for the resulting conjugates. For example, the specific ubiquitin linkage may be from G76 of the distal ubiquitin to any one of M1, K6, K11, K27, K29, K33, K48, or K63 of the proximal ubiquitin. Site specificity of attachment may be provided by both: the specific position at which each ubiquitin is fused to the remainder of the moiety that comprises said ubiquitin; and the specific ubiquitin linkage. The specific ubiquitin linkage is typically provided by selection of a specific E2 enzyme or E2/E3 enzyme combination that catalyses formation of the desired linkage, preferably in combination with ensuring that the distal ubiquitin cannot act as a proximal ubiquitin and also that the proximal ubiquitin cannot act as a distal ubiquitin, thereby ensuring only conjugates comprising the intended distal moiety conjugated to proximal moiety by the desired linkage are provided. For example, in order to prevent a distal moiety forming a ubiquitin linkage with another distal moiety, the distal ubiquitin of the distal moiety may comprise a mutation that removes the primary amine at the position where the where the E2 or E2/E3 enzyme combination forms the ubiquitin linkage; e.g. where the linkage is G76 to K48, the distal moiety may comprise K48X, where X is selected from A, R or C. For example, in order to prevent a proximal moiety forming a ubiquitin linkage with another proximal moiety, the proximal ubiquitin of the proximal moiety may comprise a blocked C-terminus. Exemplary combinations of mutations and E2 or E2/E3 enzymes that provide specific ubiquitin linkages are provided in Table 2.

TABLE 2
Mutation and enzyme combinations to
provide specific ubiquitin linkages

| Ubiquitin | Distal | | |
|---|---|---|---|
| linkage | ubiquitin | | By- |
| type | mutation(s) | E2 or E2/E3 enzymes | product(s)⁽⁹⁾ |
| K63 | K63A, K63C | Mms2 and Ubc13⁽¹⁾ | |
| | or K63R | Ubc13 and Uev1A⁽²⁾ | |
| K48 | K48A, K48C | Ubc7-gp78RING | |
| | or K48R | fusion^{(2a)} | |
| | | (Vincent Chau lab) | |
| | | E2: 25K (UbcH1)⁽¹⁾ | |
| | | E2: Ccd34⁽²⁾ | |
| K33 | | E2: UBE2D1 (UbcH5a), | K11, K48, |
| | | AREL1 ⁽³⁾ | K63, K6 |
| | | E2: UBE2L3 (UbcH7), | (can be |
| | | AREL1 ⁽⁴⁾ | hydrolysed by |
| | | | DUBS) |
| K29 | K48A, K48C | UBE2D3 and UBE3C⁽⁵⁾ | K48 |
| | or K48R | | (can be |
| | | | hydrolysed by |
| | | | DUBS) |
| K11 | Double mutation: | E2: UbE2S ⁽⁶⁾ | K63 |
| | K11R-K63R | UbE2S-UBD fusion (E2 | (can be |
| | for distal | fused to a ubiquitin | hydrolysed by |
| | ubiquitin; | binding domain) ⁽⁷⁾ | DUBS) |
| | with K63R-D77 | | |
| | for proximal | | |
| | ubiquitin | | |
| K6 | K6R-K48R | NleL and UbcH7 ⁽⁸⁾ | K48 |
| | with K48R- | | (can be |
| | ΔG76 | | hydrolysed by |
| | | | DUBS) |

⁽¹⁾Pickart C M, Raasi S. Controlled synthesis of polyubiquitin chains. Methods Enzymol. 2005; 399: 21-36.
⁽²⁾Komander D, et al. The structure of the CYLD USP domain explains its specificity for Lys63-linked polyubiquitin and reveals a B box module. Mol Cell. 2008; 29(4): 451-64.
^{(2a)}K. Davidshofer, “Allosteric activation of ubiquitin conjugating enzymes by RING domains”, PhD dissertation, 2008, available at url https://etda.libraries.psu.edu/catalog/8500
⁽³⁾ Kristariyanto YA, et al. Assembly and structure of Lys33-linked polyubiquitin reveals distinct conformations. Biochem J. 2015; 467(2): 345-52.
⁽⁴⁾ Michel MA, et al. Assembly and specific recognition of k29- and k33-linked polyubiquitin. Mol Cell. 2015; 58(1): 95-109.
⁽⁵⁾Kristariyanto Y A, et al. K29-selective ubiquitin binding domain reveals structural basis of specificity and heterotypic nature of k29 polyubiquitin. Mol Cell. 2015; 58(1): 83-94.
⁽⁶⁾ Castaneda C A, et al. Unique structural, dynamical, and functional properties of k11-linked polyubiquitin chains. Structure. 2013; 21(7): 1168-81.
⁽⁷⁾ Bremm A, et al. Lys11-linked ubiquitin chains adopt compact conformations and are preferentially hydrolyzed by the deubiquitinase Cezanne. Nat Struct Mol Biol. 2010; 17(8): 939-47.
⁽⁸⁾ Hospenthal M K, et al. Assembly, analysis and architecture of atypical ubiquitin chains. Nat Struct Mol Biol. 2013; 20(5): 555-65.
⁽⁹⁾ Examples of suitable DUBs are set out in Table 3.

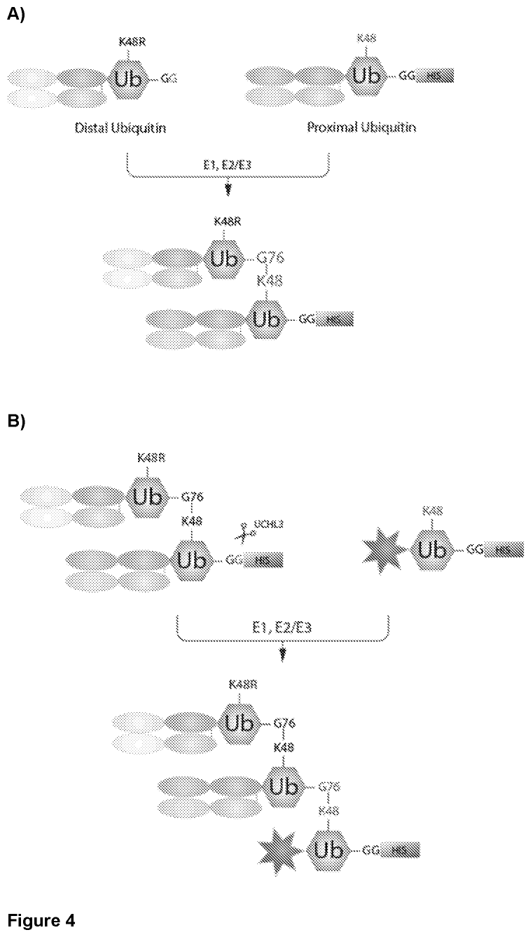

FIG. 4 provides an example of the present methods that provide site specificity for (A) a conjugate that is a UbiFab dimer and (B) a conjugate that is a trimer comprising a UbiFab dimer further conjugated with a ubiquitin substituted with a probe. FIG. 4A provides a schematic illustrating the formation of a UbiFab dimer from a distal UbiFab and a proximal UbiFab. The enzymes E2 and (when present) E3 are selected such that the ubiquitin linkage is from G76 of the distal ubiquitin to K48 of the proximal ubiquitin. The distal UbiFab is a distal moiety comprising an antigen binding antibody fragment and the distal ubiquitin at its C-terminus. The distal ubiquitin comprises a mutation K48R, which means that it cannot act as a proximal ubiquitin with the selected E2/E3 enzyme system. The proximal UbiFab is a proximal moiety comprising an antigen binding antibody fragment and the proximal ubiquitin at its C-terminus. The C-terminus of the proximal ubiquitin is blocked by a His-tag, which means that the proximal ubiquitin cannot act as a distal ubiquitin. The only conjugate product produced by the is reaction is the distal UbiFab conjugated to the proximal UbiFab by distal ubiquitin G76 to proximal ubiquitin K48, due to the specificity of the enzyme conjugation and the structures of the linked ubiquitins. The conjugate thus has a specific structure and high degree of homogeneity.

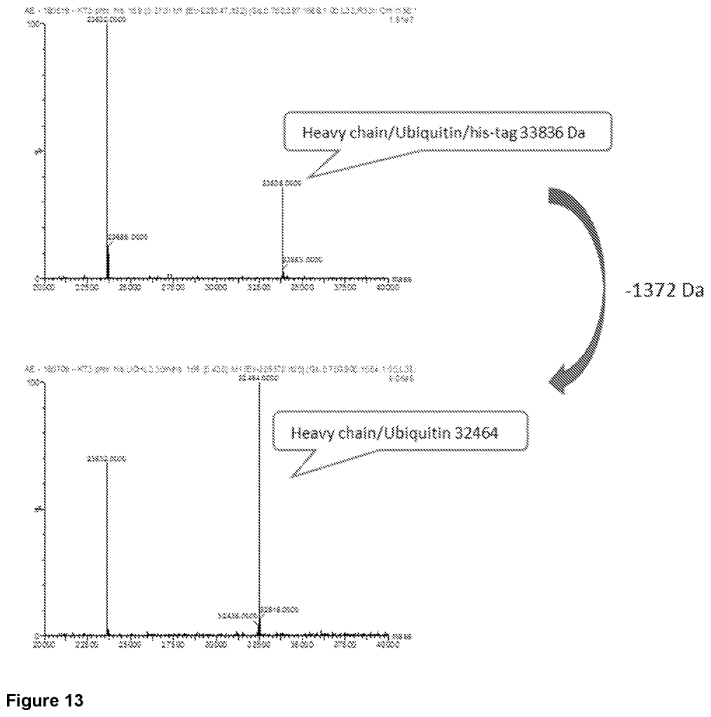

The C-terminus could be blocked in another manner as disclosed herein, other than by using a His-tag. For example, G76 may be deleted or substituted with another amino acid. Provision of the proximal ubiquitin with a C-terminus blocked with a His-Tag may, however, provide a number of advantages. For example, the His-tag may provide a handle for purification, e.g. of the proximal ubiquitin prior to conjugation and/or of the conjugate formed. The His-Tag may assist in visualisation; e.g. by western blotting using an anti-his antibody. The His-tag may be efficiently cleaved from the C-terminus of the proximal ubiquitin using a suitable DUB, such as UCHL3 (see FIG. 13). This provides an exposed C-terminus, e.g. allowing the dimer conjugate to be elongated into a trimer conjugate.

This elongation is illustrated in schematic form in FIG. 4B. As a first step, the His-tag is cleaved from the C-terminus of the proximal ubiquitin of the UbiFab dimer conjugate using a suitable DUB. The ubiquitin dimer, with its proximal ubiquitin comprising an exposed C-terminus, could now be considered to represent a distal moiety comprising a distal ubiquitin available for conjugation with a proximal ubiquitin. The ‘distal ubiquitin’ of this distal moiety is the proximal ubiquitin with an exposed C-terminus. The other portions of the dimer conjugate (e.g. its distal UbiFab and the FAB moiety of its proximal UbiFab) form part of the ‘distal moiety’. The ‘distal moiety’ can be reacted with a further ‘proximal moiety’, e.g. using the same enzyme system for forming a ubiquitin linkage from G76 of the distal ubiquitin to K48 of the proximal ubiquitin as the enzyme system used for the reaction of FIG. 4A. As illustrated in FIG. 4B, the ‘proximal moiety’ is a probe substituted ubiquitin that comprises a His-tag blocked C-terminus, but an available K48. The ‘distal moiety’ comprises an available G76, but does not have an available K48, because the ‘distal moiety's’ distal ubiquitin comprises K48R, while its proximal ubiquitin's K48 is already linked to the G76 of its distal ubiquitin. As further illustrated in FIG. 4B, The only conjugate product produced by the is reaction is the distal UbiFab dimer conjugated to the proximal probe substituted ubiquitin by ‘distal moiety’ proximal ubiquitin G76 to ‘proximal moiety’ proximal ubiquitin K48, due to the specificity of the enzyme conjugation and the structures of the linked ubiquitins. The trimer conjugate thus has a specific structure and high degree of homogeneity.

The method provided in FIG. 4B provides one way of forming a trimer conjugate with a specific structure. This method has the added advantage that the conjugate may be readily elongated to larger multimers in a stepwise manner: cleaving the His-tag from the C-terminus of the ubiquitin of the ‘proximal moiety’ of the trimer conjugate allows the trimer conjugate to provide a distal moiety for reaction with a further proximal moiety comprising a ubiquitin with a His-tag, generating a tetramer conjugate. This process may be repeated with each larger conjugate, until a multimer with the desired number of moieties comprising a ubiquitin is provided. The multimer conjugates produced by this method have a specific structure and high degree of homogeneity. In addition, the approach is readily scalable to provide larger conjugates that comprise ubiquitin multimers. There are also other approaches that may be used to provide conjugates in accordance with the present disclosure.

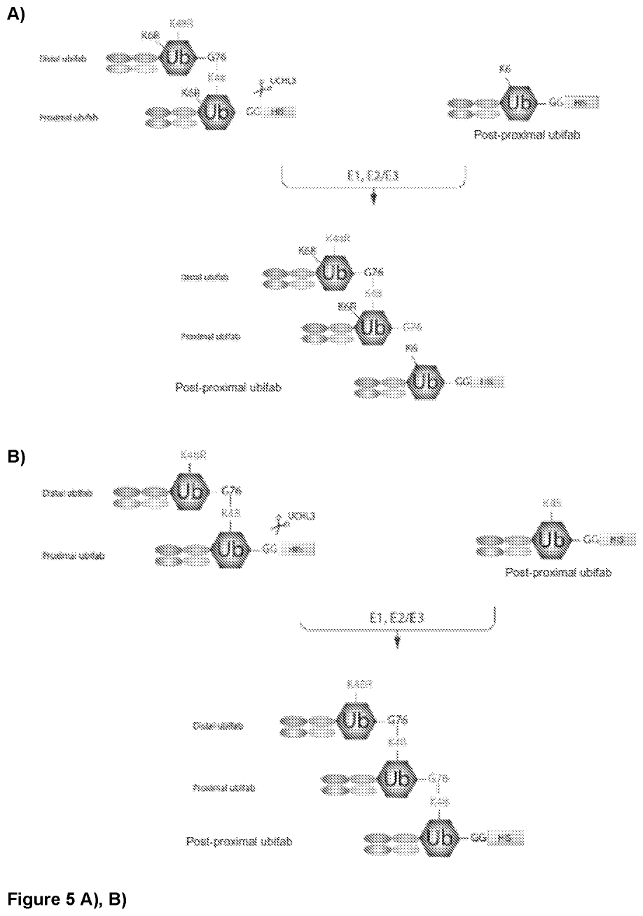

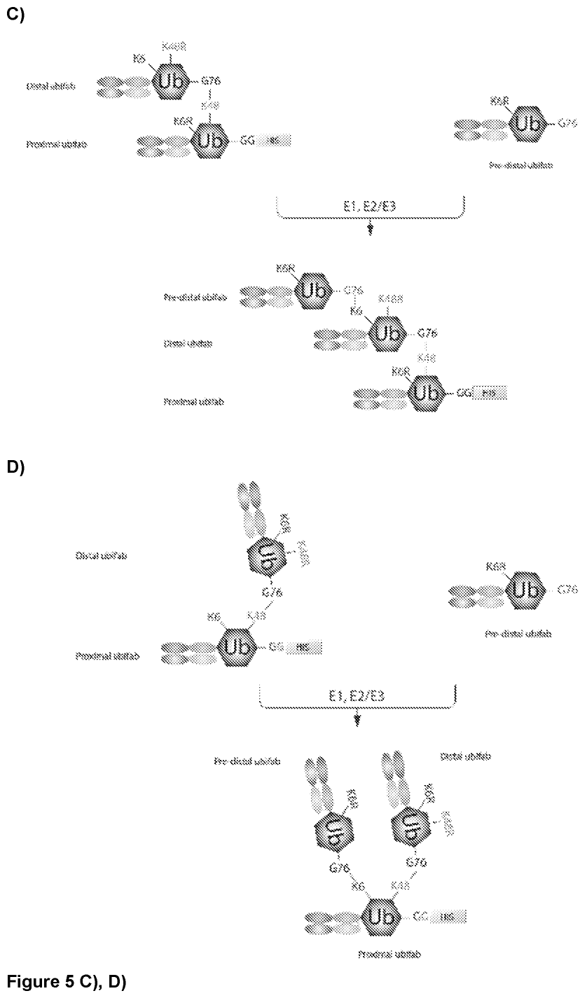

FIG. 5 illustrates four methods that may be used to provide conjugates that comprise ubiquitin multimers (e.g. ubiquitin trimers). For ease of reference, each of the initial dimers will be considered to provide a distal UbiFab and a proximal UbiFab and the further UbiFab added to the dimer to form the conjugate comprising a ubiquitin trimer will be termed either a pre-distal UbiFab or a post-proximal UbiFab. It should, however, be understood that in reactions between a pre-distal UbiFab and ubiquitin dimer, the pre-distal UbiFab comprises the distal ubiquitin and the ubiquitin dimer comprises the proximal ubiquitin; while in a reaction between a post-proximal UbiFab and ubiquitin dimer, the ubiquitin dimer comprises the distal ubiquitin and the post-proximal UbiFab comprises the proximal ubiquitin.

In FIG. 5A, a post-proximal UbiFab is conjugated to a UbiFab dimer using a different ubiquitin linkage type as the ubiquitin linkage type used in the dimer. In this example, similarly to the method of FIG. 4B, the first step involves use of a DUB to cleave the His-tag from the proximal ubiquitin of the proximal UbiFab. The second step involves the use of an E1 and E2 (or E1 and E2/E3) enzyme system, with the E2 (or E2/E3) enzymes selected to catalyse a G76 to K6 linkage, to form the conjugate comprising a ubiquitin trimer. In order to ensure that the UbiFab dimer cannot act as a ‘proximal ubiquitin’ with the selected E2/E3 enzyme system, both the distal ubiquitin and the proximal ubiquitin comprise a mutation K6R. The distal ubiquitin also comprises a second mutation, K48R, which was provided to ensure it could only be a distal ubiquitin in the reaction that formed the dimer (the dimer has a linkage distal ubiquitin G76 to proximal ubiquitin K48). The post-proximal ubiquitin of the post-proximal UbiFab comprises a His-tag blocked C-terminus. The trimer conjugate thus has a specific structure and high degree of homogeneity.

In FIG. 5B, a post-proximal UbiFab is conjugated to a UbiFab dimer using the same ubiquitin linkage type as the ubiquitin linkage type used in the dimer. The reaction illustrated here is similar to that provided in FIG. 4B, with the main difference being that in FIG. 4B the post-proximal moiety is a probe substituted ubiquitin, while in FIG. 5B the post-proximal moiety is a UbiFab. This illustrates that the illustrated methods work with any suitable ubiquitin moiety disclosed herein. As illustrated, the conjugate formed in the method illustrated in FIG. 5B comprises a ubiquitin trimer with a specific structure and high degree of homogeneity.

In FIG. 5C, a pre-distal UbiFab is conjugated to a UbiFab dimer using a different ubiquitin linkage type as the ubiquitin linkage type used in the dimer. The linkage in the dimer is distal ubiquitin G76 to proximal ubiquitin K48, while the linkage in the further step of forming the trimer is pre-distal ubiquitin G76 to distal ubiquitin K6, catalysed by appropriate enzyme selection. In order to ensure that the dimer was formed with the intended structure, the distal ubiquitin comprises a mutation K48R and the proximal ubiquitin comprises a His-Tag blocked C-terminus. In order to ensure that the trimer is formed with the correct structure, the pre-distal ubiquitin and proximal ubiquitin comprise a mutation K6R and the proximal ubiquitin comprises blocked C-terminus. As illustrated, the conjugate formed in the method illustrated in FIG. 5C comprises a ubiquitin trimer with a specific structure and high degree of homogeneity.

In FIG. 5D, a pre-distal UbiFab is conjugated to the proximal ubiquitin of a UbiFab dimer using a different ubiquitin linkage type to the ubiquitin linkage type used in the dimer. The reaction illustrated here and enzymes used are similar to those provided in FIG. 5C. The main difference is that in FIG. 5C the pre-distal ubiquitin and proximal ubiquitin comprise a mutation K6R, while in FIG. 5D it is the pre-distal ubiquitin and distal ubiquitin comprise a mutation K6R. This means that in FIG. 5D, the linkage in the further step of forming the trimer is pre-distal ubiquitin G76 to proximal ubiquitin K6, catalysed by appropriate enzyme selection; as only the proximal ubiquitin has a K6 available to form the ubiquitin linkage. The resulting trimer thus has both the distal ubiquitin and pre-distal ubiquitin conjugated directly to the proximal ubiquitin. As illustrated, the conjugate formed in the method illustrated in FIG. 5D comprises a ubiquitin trimer with a specific structure and high degree of homogeneity.

For ease of reference, FIGS. 4 and 5 have illustrated the chemistry using moieties that are UbiFabs and/or moieties that comprise a ubiquitin substituted by a probe. As the skilled person will appreciate, this chemistry and methodology is also applicable to other moieties comprising ubiquitin disclosed herein, such as fusion proteins comprising a ubiquitin, UbiMabs, etc. Accordingly, various ubiquitin dimers and multimers may be made in accordance with the methods disclosed herein.

As the skilled person will appreciate, higher order multimers may be formed by combining these methodologies. The key requirements in methods of forming higher order multimers with a specific structure and high degree of homogeneity, when using a suitable enzyme system (where E2 or E2/E3 are selected to provide a specific ubiquitin linkage) are that:
(a) the moiety comprising the ‘distal ubiquitin’ (which may be a conjugate) comprises:
   a. a single exposed ubiquitin C-terminus; and
   b. due to mutations and/or existing amide bonds, does not have any ubiquitins with a primary amine on the residue (i.e. N terminus M1, or sidechain of one of K6, K11, K27, K29, K33, K48, or K63) targeted for the linkage by the enzyme system; and
(b) the moiety comprising the ‘proximal ubiquitin’ for the reaction (which may be a conjugate):
   a. does not comprise an exposed ubiquitin C-terminus; and
   b. only comprises a single ubiquitin with a primary amine on the residue (i.e. N terminus M1, or sidechain of one of K6, K11, K27, K29, K33, K48, or K63) targeted for the linkage by the enzyme system.

The moieties comprising ubiquitin can be produced by adapting methods known in the art. For example, fusion proteins that comprise ubiquitin may be produced in transgenic cells and isolated by standard methods. The transgenic host cells may be eukaryotic cells, for example mammalian cells such as Chinese hamster ovary (CHO) cells, hybridoma cells, NSO murine myeloma cells, and PER.C6 human cells; e.g. CHO cells. The transgenic host cells may be prokaryotic cells, e.g. bacterial cells. The fusion proteins that comprise ubiquitin may be UbiFabs and/or UbiMabs. Examples of production systems for recombinant antibodies, which may be adapted for fusion protein that comprise ubiquitin, are set out in Schirrmann T., et al., Front Biosci. (2008), 13:4576-94. Review. Other expression systems that may be adapted to provide fusion proteins that comprise ubiquitin include the methods disclosed in, e.g.: Köhler, G. & Milstein, C., Nature (1975) 256, 495-497; Cheong, T.-C., et al., Nat. Commun., (2016) 7, 10934; Pogson, M., et al., Nat. Commun., (2016), 7, 12535; and Mason, D. M. et al. High-throughput antibody engineering in mammalian cells by CRISPR/Cas9-mediated homology-directed mutagenesis. Nucleic Acids Res. (2018). doi:10.1093/nar/gky550.

We have identified that, where transgenic host cells are used to express a proximal ubiquitin comprising a blocked terminus comprising a His-tag, the His-tag may be cleaved off the expressed protein. Without wishing to be bound by any theory, it is believed that this may be due to the release of DUBs from cultured cells. Addition of a selective DUB inhibitor to the medium significantly reduced cleavage off the His-tag. Where a polypeptide comprising a His-tagged proximal ubiquitin is being expressed, a selective DUB inhibitor (e.g. C-terminally propargylated ubiquitin (Ub-PA)) is preferably added to the culture medium.

The ubiquitin moieties may be prepared from synthetic ubiquitin. Synthetic ubiquitin may be prepared by total linear synthesis using solid phase peptide synthesis, e.g. as described in F. El Oualid, et al., Angew. Chem. Int. Ed. Engl., (2010), 49(52), 10149-53; or D. S. Hameed, et al., Bioconjug Chem., (2017), 28(3), 805-15. The use of solid phase synthesis allows the introduction of chemical moieties at the C- or N-terminus, as well as the incorporation of unnatural amino acids at any position. The chemical moieties added to the C- or N-terminus may comprise a probe. Unnatural amino acids may comprise a probe. A synthetic ubiquitin may therefore provide a ubiquitin substituted with a probe.

An embodiment provides a method for the production of a conjugate. The method comprises:

(i) providing a solution comprising a distal moiety, a proximal moiety, a ubiquitin activating enzyme (E1), a ubiquitin-conjugating enzyme (E2) and optionally a ubiquitin-ligating enzyme (E3); and

(ii) forming a first conjugate.

The distal moiety comprises a distal ubiquitin at its C-terminus, the distal ubiquitin optionally comprising at least one of the following mutations: K6X, K11X, K27X, K29X, K33X, K48X, K63X, or K63X, where X is selected from R, A or C. The proximal moiety comprises a polypeptide comprising either a proximal ubiquitin at its C-terminus or a proximal ubiquitin at its N-terminus; said ubiquitin comprising a blocked C-terminus. The first conjugate is formed such that the distal moiety is conjugated to the proximal moiety via an amide bond from G76 of the distal ubiquitin to one of M1, K6, K11, K27, K29, K33, K48, or K63 of the proximal ubiquitin. In an embodiment, the solution comprises a ubiquitin-conjugating enzyme (E2) but does not comprise a ubiquitin-ligating enzyme (E3). In an embodiment, the solution comprises a ubiquitin-conjugating enzyme (E2) and a ubiquitin-ligating enzyme (E3).

The E2 or E2/E3 enzymes are selected for the desired ubiquitin linkage type, i.e. G76 to M1, G76 to K6, G76 to K11, G76 to K27, G76 to K33, G76 to K48, G76 to K63; e.g. for G76 to K48. Exemplary E2 or E2/E3 enzymes for the desired ubiquitin linkage type may be selected in accordance with Table 2. For example, the E2 and E3 enzymes may be Mms2 and Ubc13, or Ubc13 and Uev1A. For example, to link G76 to K48, the E2 and E3 enzyme may be UbcH7 and Gp78, e.g. the K48 linkage specific E2/E3 fusion gp78RING-Ubc7. For example, to link K6 and K48, the the E2 and E3 enzymes UbcH7 and NIeL may be used.

In an embodiment where the distal moiety is conjugated to the proximal moiety via an amide bond from G76 of the distal ubiquitin to M1 of the proximal ubiquitin; the distal moiety may not comprise any of the mutations K6X, K11X, K27X, K29X, K33X, K48X, K63X, or K63X, where X is selected from R, A or C. In an embodiment where the distal moiety is conjugated to the proximal moiety via an amide bond from G76 of the distal ubiquitin to one of K6, K11, K27, K29, K33, K48, or K63 of the proximal ubiquitin; the distal ubiquitin comprises a mutation K to X, where the mutation is at the K position in the distal ubiquitin corresponding to the K position of the proximal moiety involved in the amide bond.

Where the distal moiety is conjugated to the proximal moiety via an amide bond from G76 of the distal ubiquitin to one of K6, K11, K27, K29, K33, K48, or K63 of the proximal ubiquitin; the distal ubiquitin comprises a mutation at its corresponding lysine, e.g. as set out in Table 1. Where the distal moiety is conjugated to the proximal moiety via an amide bond from G76 of the distal ubiquitin to M1 of the proximal ubiquitin; the distal moiety may not comprise any of the mutations K6X, K11X, K27X, K29X, K33X, K48X, K63X, or K63X, where X is selected from R, A or C. Where the distal moiety is conjugated to the proximal moiety via an amide bond from G76 of the distal ubiquitin to K6 of the proximal ubiquitin; the distal ubiquitin comprises the mutation K6X. Where the distal moiety is conjugated to the proximal moiety via an amide bond from G76 of the distal ubiquitin to K11 of the proximal ubiquitin; the distal ubiquitin comprises the mutation K11x. Where the distal moiety is conjugated to the proximal moiety via an amide bond from G76 of the distal ubiquitin to K27 of the proximal ubiquitin; the distal ubiquitin comprises the mutation K27X. Where the distal moiety is conjugated to the proximal moiety via an amide bond from G76 of the distal ubiquitin to K29 of the proximal ubiquitin; the distal ubiquitin comprises the mutation K29X. Where the distal moiety is conjugated to the proximal moiety via an amide bond from G76 of the distal ubiquitin to K33 of the proximal ubiquitin; the distal ubiquitin comprises the mutation K33X. Where the distal moiety is conjugated to the proximal moiety via an amide bond from G76 of the distal ubiquitin to K48 of the proximal ubiquitin; the distal ubiquitin comprises the mutation K48X. Where the distal moiety is conjugated to the proximal moiety via an amide bond from G76 of the distal ubiquitin to K48 of the proximal ubiquitin; the distal ubiquitin comprises the mutation K48X. Where the distal moiety is conjugated to the proximal moiety via an amide bond from G76 of the distal ubiquitin to K63 of the proximal ubiquitin; the distal ubiquitin comprises the mutation K63X.

The distal moiety may be conjugated to the proximal moiety via an amide bond from G76 of the distal ubiquitin to K6, K48 or K63 of the proximal ubiquitin. For example, the distal moiety may be conjugated to the proximal moiety via an amide bond from G76 of the distal ubiquitin to K6 or K48 of the proximal ubiquitin; or the distal moiety may be conjugated to the proximal moiety via an amide bond from G76 of the distal ubiquitin to K48 or K63 of the proximal ubiquitin. The distal moiety may be conjugated to the proximal moiety via an amide bond from G76 of the distal ubiquitin to K48 of the proximal ubiquitin.

The distal ubiquitin may comprise the mutation K48R or K480, wherein the amide bond is from G76 of the distal ubiquitin to K48 of the proximal ubiquitin.

The proximal moiety may comprise a polypeptide comprising a proximal ubiquitin at its C-terminus, said ubiquitin comprising a blocked C-terminus; wherein the distal moiety is conjugated to the proximal moiety via an amide bond from G76 of the distal ubiquitin to one of K6, K11, K27, K29, K33, K48, or K63 of the proximal ubiquitin. Providing a proximal ubiquitin having a blocked C-terminus is advantageous, as this prevents the proximal ubiquitin acting as a distal ubiquitin in the methods of the invention.

The proximal ubiquitin may comprise a blocked C-terminus. For example, the blocked C-terminus may comprise a deleted G76, or a G76-Z, where —Z is a sequence of one or more amino acids (e.g. 2-20 amino acids). The blocked C-terminus may comprise a deleted G76. The blocked C-terminus may comprise a G76-Z. The -Z may be a polyhistidine (His-tag), e.g. comprising at least 4 amino acids, such as a hexa histidine-tag.

The distal moiety may comprise a fusion protein with ubiquitin at its C-terminus. The distal moiety may comprise a distal ubiquitin substituted with a probe. The proximal moiety may comprise a fusion protein with ubiquitin at its C-terminus. The proximal moiety may comprise a fusion protein with ubiquitin at its N-terminus. The proximal moiety may comprise a proximal ubiquitin substituted with a probe. The distal moiety and the proximal moiety may both independently comprise a fusion protein with ubiquitin at its C-terminus; or one of the distal moiety and the proximal moiety may comprise a fusion protein and the other of the distal moiety and the proximal moiety may comprise a ubiquitin substituted with a probe.

The distal moiety may comprise a fusion protein comprising an antigen binding antibody fragment, such as a Fab, and the distal ubiquitin at its C-terminus (e.g. UbiFab), or a fusion protein comprising a monoclonal antibody and the distal ubiquitin at the C-terminus of the heavy chain or the light chain of the monoclonal antibody (UbiMab). The distal moiety may comprise a UbiFab. The distal moiety may comprise a UbiMab. The distal moiety may comprise a UbiMab where the distal ubiquitin is at the C-terminus of the heavy chain of the monoclonal antibody. The distal moiety may comprise a UbiMab where the distal ubiquitin is at the C-terminus of the light chain of the monoclonal antibody.

The proximal moiety may comprise a fusion protein comprising an antigen binding antibody fragment and the proximal ubiquitin at its C-terminus or N-terminus (UbiFab), or a fusion protein comprising a monoclonal antibody and the distal ubiquitin at the C-terminus (or N-terminus) of the heavy chain or the light chain of the monoclonal antibody (UbiMab). The proximal moiety may comprise a UbiFab where the proximal ubiquitin is at its C-terminus. The proximal moiety may comprise a UbiFab where the proximal ubiquitin is at its N-terminus. The proximal moiety may comprise a UbiMab. The proximal moiety may comprise a UbiMab where the proximal ubiquitin is at the C-terminus (or N-terminus) of the heavy chain of the monoclonal antibody. The proximal moiety may comprise a UbiMab where the proximal ubiquitin is at the C-terminus (or N-terminus) of the light chain of the monoclonal antibody.

Where the distal moiety comprises a fusion protein, providing the distal moiety may comprise expressing the distal fusion protein in a cell and isolating the distal fusion protein. The cell may be a eukaryotic cell, for example a mammalian cell such as a Chinese hamster ovary (CHO) cell, a hybridoma cell, a NSO murine myeloma cell, and a PER.C6 human cell; e.g. a CHO cell. The cell may be a CHO cell or a hybridoma cell. The cell may be a CHO cell. The cell may be a hybridoma cell. The cell may be a prokaryotic cell, such as a bacterial cell. The cell may be a transgenic cell, e.g. comprising an inserted gene encoding the fusion protein.

Where the proximal moiety comprises a fusion protein, providing the proximal moiety may comprise expressing the proximal fusion protein in a cell and isolating the proximal fusion protein. The cell may be a eukaryotic cell, for example a mammalian cell such as a Chinese hamster ovary (CHO) cell, a hybridoma cell, a NSO murine myeloma cell, and a PER.C6 human cell. The cell may be a CHO cell or a hybridoma cell. The cell may be a CHO cell. The cell may be a hybridoma cell. The cell may be a prokaryotic cell, such as a bacterial cell. The cell may be a transgenic cell, e.g. comprising an inserted gene encoding the fusion protein.

The proximal moiety may comprise a fusion protein comprising a blocked C-terminus comprising a G76-His-tag and expressing the proximal fusion protein may comprise culturing said eukaryotic cell in a medium supplemented with a deubiquitinating enzyme (DUBs) inhibitor. The DUBs inhibitor may be or comprise propargylated ubiquitin (Ub-PA).

Where the distal moiety comprises a distal ubiquitin substituted with a probe, the probe may be a payload or a label. Providing the distal ubiquitin substituted with a probe may comprise synthesis of the ubiquitin substituted with a probe by total linear synthesis using solid phase peptide synthesis and isolating the distal ubiquitin substituted with a probe.

Where the proximal moiety comprises a proximal ubiquitin substituted with a probe, the probe may be a payload or a label. Providing the proximal ubiquitin substituted with a probe may comprise synthesis of the proximal ubiquitin substituted with a probe by total linear synthesis using solid phase peptide synthesis and isolating the proximal ubiquitin substituted with a probe.

The method may further comprise:

(iii) unblocking the blocked C-terminus of the first conjugate to provide an unblocked first conjugate.

The blocked C-terminus may comprise a G76-His-tag, wherein the unblocking comprises contacting the conjugate with a deubiquitinating enzyme (DUB). The DUB may be UCHL3.

In an embodiment, method further comprises:

(iv) providing a solution comprising the unblocked first conjugate, a post-proximal moiety, a ubiquitin activating enzyme (E1), a ubiquitin-conjugating enzyme (E2) and a optionally a ubiquitin-ligating enzyme (E3);

wherein the post-proximal moiety comprises a polypeptide comprising a post-proximal ubiquitin at its C-terminus, the post-proximal ubiquitin comprising a blocked C-terminus, or a post-proximal ubiquitin at its N-terminus; and

(v) thereby forming a second conjugate such that the unblocked first conjugate is conjugated to the post-proximal moiety via an amide bond from G76 of the proximal ubiquitin to one of M1, K6, K11, K27, K29, K33, K48, or K63 of the post-proximal ubiquitin. In an embodiment, the solution comprises a ubiquitin-conjugating enzyme (E2) but does not comprise a ubiquitin-ligating enzyme (E3). In an embodiment, the solution comprises a ubiquitin-conjugating enzyme (E2) and a ubiquitin-ligating enzyme (E3).

The E2 or E2/E3 enzymes for the solution of (iv) are selected for the desired ubiquitin linkage type, i.e. G76 to M1, G76 to K6, G76 to K11, G76 to K27, G76 to K33, G76 to K48, G76 to K63; e.g. for G76 to K48. Exemplary E2 or E2/E3 enzymes for the desired ubiquitin linkage type may be selected in accordance with Table 2. For example, the E2 and E3 enzymes may be Mms2 and Ubc13, or Ubc13 and Uev1A. For example, to link G76 to K48, the E2 and E3 enzyme may be UbcH7 and Gp78, e.g. the K48 linkage specific E2/E3 fusion gp78RING-Ubc7. For example, to link K6 and K48, the the E2 and E3 enzymes UbcH7 and NIeL may be used.

In an embodiment where the unblocked first conjugate is conjugated to the post-proximal moiety via an amide bond from G76 of the proximal ubiquitin to M1 of the M1 of the post-proximal ubiquitin; the distal ubiquitin and proximal ubiquitin do not have an exposed N-terminus (M1 residue). In an embodiment where the unblocked first conjugate is conjugated to the post-proximal moiety via an amide bond from G76 of the proximal ubiquitin to one of K6, K11, K27, K29, K33, K48, or K63 of the post-proximal ubiquitin; neither the distal ubiquitin nor the proximal ubiquitin comprises an available lysine primary amine at the K position in the distal ubiquitin and proximal ubiquitin corresponding to the K position of the post-proximal moiety involved in the amide bond. Each relevant ubiquitin may independently lack a lysine primary amine due to a mutation K to X, where X is selected from R, A or C; or each relevant ubiquitin may independently lack a lysine primary amine due to an amide bond (for example a ubiquitin linkage between the distal ubiquitin and the proximal ubiquitin).

The unblocked first conjugate may be conjugated to the post-proximal moiety via an amide bond from G76 of the proximal ubiquitin to K6, K48 or K63 of the post-proximal ubiquitin. For example, the unblocked first conjugate may be conjugated to the post-proximal moiety via an amide bond from G76 of the proximal ubiquitin to K6 or K48 of the post-proximal ubiquitin; or the first unblocked conjugate may be conjugated to the post-proximal moiety via an amide bond from G76 of the proximal ubiquitin to K48 or K63 of the post-proximal ubiquitin. The first unblocked conjugate may be conjugated to the post-proximal moiety via an amide bond from G76 of the proximal ubiquitin to K48 of the post-proximal ubiquitin.

The distal ubiquitin may comprise the mutation K48R or K48C and the ubiquitin linkage in the first unblocked conjugate may be G76 to K48, wherein the amide bond formed in step (v) is from G76 of the proximal ubiquitin to K48 of the post-proximal ubiquitin. Both the distal ubiquitin and proximal ubiquitin may comprise the mutation K48R or K48C, wherein the amide bond formed in step (v) is from G76 of the proximal ubiquitin to K48 of the post-proximal ubiquitin.

The post-proximal moiety may comprise a polypeptide comprising a post-proximal ubiquitin at its C-terminus, said ubiquitin comprising a blocked C-terminus; wherein the first unblocked conjugate is conjugated to the post-proximal moiety via an amide bond from G76 of the proximal ubiquitin to one of K6, K11, K27, K29, K33, K48, or K63 of the post-proximal ubiquitin. Providing a post-proximal ubiquitin having a blocked C-terminus is advantageous, as this prevents the post-proximal ubiquitin acting as a distal ubiquitin in the methods of the invention.

The post-proximal ubiquitin may comprise a blocked C-terminus. For example, the blocked C-terminus may comprise a deleted G76, or a G76-Z, where —Z is a sequence of one or more amino acids (e.g. 2-20 amino acids). The blocked C-terminus may comprise a deleted G76. The blocked C-terminus may comprise a G76-Z. The -Z may be a polyhistidine (His-tag), e.g. comprising at least 4 amino acids, such as a hexa histidine-tag.

The post-proximal moiety may comprise a fusion protein comprising an antigen binding antibody fragment and the proximal ubiquitin at its C-terminus or N-terminus (UbiFab), or a fusion protein comprising a monoclonal antibody and the distal ubiquitin at the C-terminus (or N-terminus) of the heavy chain or the light chain of the monoclonal antibody (UbiMab). The post-proximal moiety may comprise a UbiFab where the post-proximal ubiquitin is at its C-terminus. The post-proximal moiety may comprise a UbiFab where the post-proximal ubiquitin is at its N-terminus. The post-proximal moiety may comprise a UbiMab. The post-proximal moiety may comprise a UbiMab where the proximal ubiquitin is at the C-terminus (or N-terminus) of the heavy chain of the monoclonal antibody. The post-proximal moiety may comprise a UbiMab where the proximal ubiquitin is at the C-terminus (or N-terminus) of the light chain of the monoclonal antibody.

Where the post-proximal moiety comprises a fusion protein, providing the post-proximal moiety may comprise expressing the proximal fusion protein in a cell and isolating the post-proximal fusion protein. The cell may be a eukaryotic cell, for example a mammalian cell such as a Chinese hamster ovary (CHO) cell, a hybridoma cell, a NSO murine myeloma cell, and a PER.C6 human cell. The cell may be a CHO cell or a hybridoma cell. The cell may be a CHO cell. The cell may be a hybridoma cell. The cell may be a prokaryotic cell, such as a bacterial cell. The cell may be a transgenic cell, e.g. comprising an inserted gene encoding the fusion protein.

The post-proximal moiety may comprise a fusion protein comprising a blocked C-terminus comprising a G76-His-tag and expressing the post-proximal fusion protein may comprise culturing said eukaryotic cell in a medium supplemented with a deubiquitinating enzyme (DUBs) inhibitor. The DUBs inhibitor may be or comprise propargylated ubiquitin (Ub-PA).

Where the post-proximal moiety comprises a post-proximal ubiquitin substituted with a probe, the probe may be a payload or a label. Providing the post-proximal ubiquitin substituted with a probe may comprise synthesis of the post-proximal ubiquitin substituted with a probe by total linear synthesis using solid phase peptide synthesis and isolating the post-proximal ubiquitin substituted with a probe.

In an embodiment, the method further comprises:

(vi) providing a solution comprising a pre-distal moiety, the first conjugate or the second conjugate, a ubiquitin activating enzyme (E1), a ubiquitin-conjugating enzyme (E2) and optionally a ubiquitin-ligating enzyme (E3);

wherein the pre-distal moiety comprises a pre-distal ubiquitin at its C-terminus, the distal ubiquitin comprising at least one of the following mutations: K6X, K11X, K27X, K29X, K33X, K48X, or K63X, where X is selected from R, A or C; and

(vii) thereby forming a third conjugate such that the pre-distal moiety is conjugated to the first conjugate or the second conjugate via an amide bond from the G76 of the pre-distal ubiquitin to one of K6, K11, K27, K29, K33, K48, or K63 of the distal ubiquitin or the proximal ubiquitin, or (if present) the post-proximal ubiquitin.

The E2 or E2/E3 enzymes for the solution of (iv) are selected for the desired ubiquitin linkage type, i.e. G76 to M1, G76 to K6, G76 to K11, G76 to K27, G76 to K33, G76 to K48, G76 to K63; e.g. for G76 to K48. Exemplary E2 or E2/E3 enzymes for the desired ubiquitin linkage type may be selected in accordance with Table 2. For example, the E2 and E3 enzymes may be Mms2 and Ubc13, or Ubc13 and Uev1A. For example, to link G76 to K48, the E2 and E3 enzyme may be UbcH7 and Gp78, e.g. the K48 linkage specific E2/E3 fusion gp78RING-Ubc7. For example, to link K6 and K48, the the E2 and E3 enzymes UbcH7 and NIeL may be used.

Where the first conjugate or second conjugate is conjugated to the pre-distal moiety via an amide bond from G76 of the pre-distal ubiquitin to one of K6, K11, K27, K29, K33, K48, or K63 of the distal ubiquitin; neither the pre-distal ubiquitin nor the proximal ubiquitin (nor the post-proximal ubiquitin, if present) comprises an available lysine primary amine at the K position in the pre-distal ubiquitin and proximal ubiquitin corresponding to the K position of the distal ubiquitin involved in the amide bond. Where the first conjugate or second conjugate is conjugated to the pre-distal moiety via an amide bond from G76 of the pre-distal ubiquitin to one of K6, K11, K27, K29, K33, K48, or K63 of the proximal ubiquitin; neither the pre-distal ubiquitin nor the distal ubiquitin (nor the post-proximal ubiquitin, if present) comprises an available lysine primary amine at the K position in the pre-distal ubiquitin and distal ubiquitin corresponding to the K position of the proximal ubiquitin involved in the amide bond between the pre-distal ubiquitin and the proximal ubiquitin. Where the second conjugate is conjugated to the pre-distal moiety via an amide bond from G76 of the pre-distal ubiquitin to one of K6, K11, K27, K29, K33, K48, or K63 of the post-proximal ubiquitin; none of the pre-distal ubiquitin, the distal ubiquitin and the proximal ubiquitin comprises an available lysine primary amine at the K position in the pre-distal ubiquitin, distal ubiquitin and proximal ubiquitin corresponding to the K position of the post-proximal ubiquitin involved in the amide bond. Each relevant ubiquitin may independently lack a lysine primary amine due to a mutation K to X, where X is selected from R, A or C; or each relevant ubiquitin may independently lack a lysine primary amine due to an amide bond (for example a ubiquitin linkage between the distal ubiquitin and the proximal ubiquitin).

The pre-distal moiety may be conjugated to the first conjugate or second conjugate via an amide bond from G76 of the pre-distal ubiquitin to K6, K48 or K63 of the distal ubiquitin. For example, the pre-distal moiety may be conjugated to the first conjugate or second conjugate via an amide bond from G76 of the pre-distal ubiquitin to K6 or K48 of the distal ubiquitin; or the pre-distal moiety may be conjugated to the first conjugate or second conjugate via an amide bond from G76 of the pre-distal ubiquitin to K48 or K63 of the distal ubiquitin. The pre-distal moiety may be conjugated to the first conjugate or second conjugate via an amide bond from G76 of the pre-distal ubiquitin to K48 of the distal ubiquitin.

The pre-distal moiety may be conjugated to the first conjugate or second conjugate via an amide bond from G76 of the pre-distal ubiquitin to K6, K48 or K63 of the proximal ubiquitin. For example, the pre-distal moiety may be conjugated to the first conjugate or second conjugate via an amide bond from G76 of the pre-distal ubiquitin to K6 or K48 of the proximal ubiquitin; or the pre-distal moiety may be conjugated to first conjugate or second conjugate via an amide bond from G76 of the pre-distal ubiquitin to K48 or K63 of the proximal ubiquitin. The pre-distal moiety may be conjugated to the first conjugate or second conjugate via an amide bond from G76 of the pre-distal ubiquitin to

K48 of the proximal ubiquitin.

The pre-distal moiety may be conjugated to the second conjugate via an amide bond from G76 of the pre-distal ubiquitin to K6, K48 or K63 of the post-proximal ubiquitin. For example, the pre-distal moiety may be conjugated to the second conjugate via an amide bond from G76 of the pre-distal ubiquitin to K6 or K48 of the post-proximal ubiquitin; or the pre-distal moiety may be conjugated to second conjugate via an amide bond from G76 of the pre-distal ubiquitin to K48 or K63 of the post-proximal ubiquitin. The pre-distal moiety may be conjugated to the second conjugate via an amide bond from G76 of the pre-distal ubiquitin to K48 of the post-proximal ubiquitin.

The pre-distal ubiquitin may comprise the mutation K48R or K48C, wherein the amide bond is from G76 of the pre-distal ubiquitin to K48 of one of the distal ubiquitin, proximal ubiquitin, or (when present) post-proximal ubiquitin. For example, The pre-distal ubiquitin may comprise the mutation K48R or K48C, wherein the amide bond is from G76 of the pre-distal ubiquitin to K48 of the distal ubiquitin.

The pre-distal moiety may comprise a fusion protein with ubiquitin at its C-terminus. The pre-distal moiety may comprise a pre-distal ubiquitin substituted with a probe. The pre-distal moiety may comprise a fusion protein comprising an antigen binding antibody fragment and the pre-distal ubiquitin at its C-terminus (UbiFab), or a fusion protein comprising a monoclonal antibody and the pre-distal ubiquitin at the C-terminus of the heavy chain or the light chain of the monoclonal antibody (UbiMab). The pre-distal moiety may comprise a UbiFab. The pre-distal moiety may comprise a UbiMab. The pre-distal moiety may comprise a UbiMab where the pre-distal ubiquitin is at the C-terminus of the heavy chain of the monoclonal antibody. The pre-distal moiety may comprise a UbiMab where the pre-distal ubiquitin is at the C-terminus of the light chain of the monoclonal antibody.

Where the pre-distal moiety comprises a fusion protein, providing the pre-distal moiety may comprise expressing the distal fusion protein in a cell and isolating the pre-distal fusion protein. The cell may be a eukaryotic cell, for example a mammalian cell such as a Chinese hamster ovary (CHO) cell, a hybridoma cell, a NSO murine myeloma cell, and a PER.C6 human cell; e.g. a CHO cell. The cell may be a CHO cell or a hybridoma cell. The cell may be a CHO cell. The cell may be a hybridoma cell. The cell may be a prokaryotic cell, such as a bacterial cell. The cell may be a transgenic cell, e.g. comprising an inserted gene encoding the fusion protein.

Where the pre-distal moiety comprises a pre-distal ubiquitin substituted with a probe, the probe may be a payload or a label. Providing the pre-distal ubiquitin substituted with a probe may comprise synthesis of the ubiquitin substituted with a probe by total linear synthesis using solid phase peptide synthesis and isolating the pre-distal ubiquitin substituted with a probe.

In a related embodiment, the method comprises steps (i) and (ii) as defined previously and further comprises the following alternative steps (iii) to (vii):

(iii) providing a solution comprising a pre-distal moiety, the first conjugate, a ubiquitin activating enzyme (E1), a ubiquitin-conjugating enzyme (E2) and optionally a ubiquitin-ligating enzyme (E3);

wherein the pre-distal moiety comprises a pre-distal ubiquitin at its C-terminus, the distal ubiquitin comprising at least one of the following mutations: K6X, K11X, K27X, K29X, K33X, K48X, or K63X, where X is selected from R, A or C; and

(iv) thereby forming a third conjugate such that the pre-distal moiety is conjugated to the first conjugate via an amide bond from the G76 of the pre-distal ubiquitin to one of K6, K11, K27, K29, K33, K48, or K63 of the distal ubiquitin or the proximal ubiquitin;

(v) unblocking the blocked C-terminus of the proximal ubiquitin of third conjugate to provide an unblocked third conjugate;

(vi) providing a solution comprising the unblocked third conjugate, a post-proximal moiety, a ubiquitin activating enzyme (E1), a ubiquitin-conjugating enzyme (E2) and optionally a ubiquitin-ligating enzyme (E3);

wherein the post-proximal moiety comprises a polypeptide comprising a post-proximal ubiquitin at its C-terminus, the post-proximal ubiquitin comprising a blocked C-terminus, or a post-proximal ubiquitin at its N-terminus; and

(vii) thereby forming a second conjugate such that the unblocked first conjugate is conjugated to the post-proximal moiety via an amide bond from G76 of the proximal ubiquitin to one of M1, K6, K11, K27, K29, K33, K48, or K63 of the post-proximal ubiquitin. In an embodiment, the solution comprises a ubiquitin-conjugating enzyme (E2) but does not comprise a ubiquitin-ligating enzyme (E3). In an embodiment, the solution comprises a ubiquitin-conjugating enzyme (E2) and a ubiquitin-ligating enzyme (E3).

The pre-distal moiety, first conjugate, E1, E2, E3, third conjugate, distal ubiquitin, proximal ubiquitin, blocked C-terminus, post-proximal moiety and second conjugate may be as further defined herein.

Where the method comprises providing more than one solution comprising E2 or E2/E3 enzymes (e.g. for steps (i) and (iv); (i) and (vi); (i), (iv) and (vi); or (i), (iii) and (vi)) the E2 or E2/E3 enzymes used in each solution may be the same. Where the method comprises providing more than one solution comprising E2 or E2/E3 enzymes (e.g. for steps (i) and (iv); (i) and (vi); (i), (iv) and (vi); or (i), (iii) and (vi)) the E2 or E2/E3 enzymes used in each solution may be different, for example to provide a different specific ubiquitin linkage type from the reaction in each solution. Where the method comprises providing at least three solutions comprising E2 or E2/E3 enzymes (e.g. for steps (i), (iv) and (vi); or (i), (iii) and (vi)) the E2 or E2/E3 enzymes used may be the same in at least two of the solutions and may be different in at least one of the solution.

While embodiments described above expressly mention methods of production of conjugates comprising up to four ubiquitins (distal ubiquitin, proximal ubiquitin, pre-distal ubiquitin, post-proximal ubiquitin) and their associated moieties, the invention is not limited to methods of forming conjugates comprising ubiquitin dimers, trimers and tetramers. Conjugates comprising ubiquitin multimers with larger numbers of ubiquitins (higher order multimers) are also contemplated. For example, in the methods disclosed herein, any one or more of the moieties may comprise a conjugate comprising a ubiquitin multimer. In an example, a ubiquitin tetramer formed according to steps (i) to (vii) disclosed herein could be subjected to a step (viii), unblocking the blocked C-terminus of the post-proximal ubiquitin of the third conjugate (or the second conjugate) to provide an unblocked third conjugate (or unblocked second conjugate) and isolating the unblocked conjugate. The unblocked conjugate (which already comprises a ubiquitin tetramer) could then act as a ‘distal moiety’ in the methods disclosed, with the post-proximal ubiquitin providing the ‘distal ubiquitin’, allowing the formation of conjugates that comprise higher order ubiquitin multimers.

In an embodiment where the conjugate is a trimer or higher order multimer, a method for the production of the multimeric conjugate may comprise:

(i) providing a solution comprising a monomeric moiety, a ubiquitin activating enzyme (E1), a ubiquitin-conjugating enzyme (E2) and optionally a ubiquitin-ligating enzyme (E3);

wherein the or each monomeric moiety comprises a ubiquitin having G-76 available for formation of an amide bond at its C-terminus; and

(ii) thereby forming the multimeric conjugate comprising at least three conjugated monomeric moieties, such that:

a first monomeric moiety is conjugated to a second monomeric moiety via an amide bond from G76 of the first monomeric moiety's ubiquitin to one of K6, K11, K27, K29, K33, K48, or K63 of the second monomeric moiety's ubiquitin; and

the second monomeric moiety is conjugated to a third monomeric moiety via an amide bond from G76 of the second monomeric moiety's ubiquitin to one of K6, K11, K27, K29, K33, K48, or K63 of the third monomeric moiety's ubiquitin.

The multimer produced may comprise 3, 4, 5, 6, 7, 8, 9, 10 or more monomeric moieties. When the multimer comprises 4 or more monomeric moieties, step (ii) may further comprise the third monomeric moiety is conjugated to a fourth monomeric moiety via an amide bond from G76 of the third monomeric moiety's ubiquitin to one of K6, K11, K27, K29, K33, K48, or K63 of the fourth monomeric moiety's ubiquitin. When the multimer comprises 5 or more monomeric moieties, step (ii) may further comprise the fourth monomeric moiety is conjugated to a fifth monomeric moiety via an amide bond from G76 of the fourth monomeric moiety's ubiquitin to one of K6, K11, K27, K29, K33, K48, or K63 of the fifth monomeric moiety's ubiquitin. When the multimer comprises 6 or more monomeric moieties, step (ii) may further comprise the fifth monomeric moiety is conjugated to a sixth monomeric moiety via an amide bond from G76 of the fifth monomeric moiety's ubiquitin to one of K6, K11, K27, K29, K33, K48, or K63 of the sixth monomeric moiety's ubiquitin. When the multimer comprises 7 or more monomeric moieties, step (ii) may further comprise the sixth monomeric moiety is conjugated to a seventh monomeric moiety via an amide bond from G76 of the sixth monomeric moiety's ubiquitin to one of K6, K11, K27, K29, K33, K48, or K63 of the seventh monomeric moiety's ubiquitin. When the multimer comprises 8 or more monomeric moieties, step (ii) may further comprise the seventh monomeric moiety is conjugated to an eighth monomeric moiety via an amide bond from G76 of the seventh monomeric moiety's ubiquitin to one of K6, K11, K27, K29, K33, K48, or K63 of the eighth monomeric moiety's ubiquitin. When the multimer comprises 9 or more monomeric moieties, step (ii) may further comprise the eighth monomeric moiety is conjugated to a ninth monomeric moiety via an amide bond from G76 of the eighth monomeric moiety's ubiquitin to one of K6, K11, K27, K29, K33, K48, or K63 of the ninth monomeric moiety's ubiquitin. When the multimer comprises 10 or more monomeric moieties, step (ii) may further comprise the ninth monomeric moiety is conjugated to a tenth monomeric moiety via an amide bond from G76 of the ninth monomeric moiety's ubiquitin to one of K6, K11, K27, K29, K33, K48, or K63 of the tenth monomeric moiety's ubiquitin. The or each monomeric moiety may further comprise any of the features described hereinabove for a distal ubiquitin.

A single monomeric moiety may be provided in the solution at step (i). When this is the case, the multimer formed in step (ii) represents a homomultimer.

The E2 or E2/E3 enzymes are selected for the desired ubiquitin linkage type, i.e. G76 to K6, G76 to K11, G76 to K27, G76 to K33, G76 to K48, G76 to K63; e.g. for G76 to K48. Exemplary E2 or E2/E3 enzymes for the desired ubiquitin linkage type may be selected in accordance with Table 2. For example, the E2 and E3 enzymes may be Mms2 and Ubc13, or Ubc13 and Uev1A. For example, to link G76 to K48, the E2 and E3 enzyme may be UbcH7 and Gp78, e.g. the K48 linkage specific E2/E3 fusion gp78RING-Ubc7. For example, to link K6 and K48, the the E2 and E3 enzymes UbcH7 and NIeL may be used.

The first monomeric moiety may be conjugated to the second monomeric moiety via an amide bond from G76 of the first monomeric moiety's ubiquitin to K6, K48 or K63 of the second monomeric moiety's ubiquitin. For example, the first monomeric moiety may be conjugated to the second monomeric moiety via an amide bond from G76 of the first monomeric moiety's ubiquitin to K48 or K63 of the second monomeric moiety's ubiquitin. The first monomeric moiety may be conjugated to the second monomeric moiety via an amide bond from G76 of the first monomeric moiety's ubiquitin to K48 of the second monomeric moiety's ubiquitin.

The second monomeric moiety may be conjugated to the third monomeric moiety via an amide bond from G76 of the second monomeric moiety's ubiquitin to K6, K48 or K63 of the third monomeric moiety's ubiquitin. For example, the second monomeric moiety may be conjugated to the third monomeric moiety via an amide bond from G76 of the second monomeric moiety's ubiquitin to K48 or K63 of the third monomeric moiety's ubiquitin. The second monomeric moiety may be conjugated to the third monomeric moiety via an amide bond from G76 of the second monomeric moiety's ubiquitin to K48 of the third monomeric moiety's ubiquitin.

Each of the subsequent monomeric moieties may be conjugated in a like manner. For example, each immediately distal monomeric moiety may be conjugated to its immediately proximate monomeric moiety via an amide bond from G76 of the immediately distal moiety's ubiquitin to K6, K48 or K63 of the immediately proximate monomeric moiety's ubiquitin. For example, the immediately distal monomeric moiety may be conjugated to the immediately proximate monomeric moiety via an amide bond from G76 of the immediately distal moiety's ubiquitin to K48 or K63 of the immediately proximate monomeric moiety's ubiquitin. The immediately distal monomeric moiety may be conjugated to the immediately proximate monomeric moiety via an amide bond from G76 of the immediately distal monomeric moiety's ubiquitin to K48 of the immediately proximate monomeric moiety's ubiquitin.

As will be appreciated, due to enzyme specificity, the ubiquitin-ubiquitin amide bonds (e.g. G76 to K48) between the monomeric moieties of the multimer will be dependent on the E2 enzyme and optional E3 enzyme included in solution (i). The E2 and E3 enzymes are Mms2 and Ubc13, or Ubc13 and Uev1A.

The or each monomeric moiety may comprise a fusion protein with the ubiquitin at its C-terminus. The or each fusion protein comprises a biologically and/or pharmaceutically active polypeptide or peptide and the ubiquitin at its C-terminus. The or each fusion protein comprises an MHC.

In an embodiment, the method further comprises:

(iii) optionally isolating the multimeric conjugate formed in step (ii);

(iv) providing a solution comprising the (optionally isolated) multimeric conjugate formed in step (ii), a distal monomeric moiety and/or a proximal monomeric moiety, a ubiquitin activating enzyme (E1), a ubiquitin-conjugating enzyme (E2) and optionally a ubiquitin-ligating enzyme (E3);

wherein the distal monomeric moiety, when present, comprises a distal ubiquitin at its C-terminus, the distal ubiquitin optionally comprising at least one of the following mutations: K6X, K11X, K27X, K29X, K33X, K48X, or K63X, where X is selected from R, A or C;

wherein the proximal monomeric moiety, when present, comprises a polypeptide comprising a proximal ubiquitin at its C-terminus, or a proximal ubiquitin at it N-terminus, said ubiquitin comprising a blocked C-terminus; and

(v) thereby forming a second multimeric conjugate such that the distal moiety, when present, is conjugated to the first monomeric moiety via an amide bond from G76 of the distal ubiquitin to one of K6, K11, K27, K29, K33, K48, or K63 of the first monomeric moiety's ubiquitin; and such that the proximal moiety, when present, is conjugated to the most proximal moiety of the multimeric conjugate formed in step (ii) via an amide bond from G76 of the ubiquitin of the most proximal moiety of the multimeric conjugate formed in step (ii) to one of K6, K11, K27, K29, K33, K48, or K63 of the proximal ubiquitin.

The distal monomeric moiety may further comprise any of the features described hereinabove for a distal moiety. The proximal monomeric moiety may further comprise any of the features described hereinabove for a proximal moiety.

The distal monomeric moiety may comprise a distal ubiquitin substituted with a probe. Where the distal monomeric moiety comprises a distal ubiquitin substituted with a probe, the probe may be a payload or a label. Providing the distal ubiquitin substituted with a probe may comprise synthesis of the ubiquitin substituted with a probe by total linear synthesis using solid phase peptide synthesis and isolating the distal ubiquitin substituted with a probe. The probe may be a label, e.g. a fluorophore.

The proximal monomeric moiety may comprise a proximal ubiquitin substituted with a probe. Where the proximal monomeric moiety comprises a proximal ubiquitin substituted with a probe, the probe may be a payload or a label. Providing the proximal ubiquitin substituted with a probe may comprise synthesis of the proximal ubiquitin substituted with a probe by total linear synthesis using solid phase peptide synthesis and isolating the proximal ubiquitin substituted with a probe. The probe may be a label, e.g. a fluorophore.

### Methods of Disassembly

Methods are also provided for the disassembly of conjugates of the disclosure. The conjugates may be disassembled by the use of one or more deubiquitinating enzymes (DUBs), which cleave the peptide bond between a ubiquitin and the moiety to which it is attached. A large number of DUBs are known in the literature, with exemplary DUBs and their mechanism of action described in A. Y. Amerik and M. Hochstrasser, Biochimica et Biophysica Acta, (2004) 1695, 189-207. The DUBs used in a method of disassembly may be relatively promiscuous (e.g. is able to cleave most or all ubiquitin linkages), or the DUBs may show an extent of linkage specificity. Exemplary DUBs that demonstrate an extent of linkage specificity or preference with regard to cleavage are set out in the Table 3. Exemplary DUBs that demonstrate an extent of linkage specificity or preference with regard to cleavage are also described in T. E. Mevissen, et al., Cell, (2013), 154(1), 169-84; P. P. Geurink, et al., Chembiochem., (2016), 17(9), 816-20; and A. G. Faesen, et al., Chem Biol., (2011), 18(12), 1550-61.

TABLE 3
Linkage specific DUBs

| Deubiquitinating Enzyme (DUB) | Linkage(s) Cleaved |
|---|---|
| AMSH | Lys63 |
| Cezanne | Lys11 |
| OTUB1 | Lys48 |
| OTUD1 | Lys63 |
| OTUD3 | Lys6, Lys11, Lys63 |
| TRABID | Lys29, Lys33, Lys63 |
| OTUD2 | Lys11, Lys27, Lys29 |
| vOTU (virus derived DUB) | Lys6, Lys11, Lys48, Lys63 |
| USP21 | Lys6, Lys11, Lys33, Lys48, Lys63 |
| OTULIN | M1 |
| TRABID | K29, K33 |
| UCHL3 | Polyhistidine (e.g. His-tag) |

Where the conjugate has a requisite activity only when its component moieties are conjugated, cleavage of a linkage may inactivate the conjugate. Where the conjugate comprises a payload, cleavage of a linkage may release the moiety comprising the payload to provide its cytotoxic effect.

In an aspect, the invention comprises a method of deactivating or cleaving a conjugate of the disclosed herein comprising contacting the conjugate with a deubiquitinating enzyme (DUB), such that a linkage between two of the moieties of the conjugate is cleaved. The linkage may be G76 to K63 and the DUB may comprise at least one of AMSH, OTUD1, OTUD3, vOTU (virus derived DUB), or USP21; for example the DUB may comprise AMSH and/or OTUD1. The linkage may be G76 to K48 and the DUB may comprise at least one of OTUB1, vOTU (virus derived DUB), USP21, or UCHL3; for example the DUB may comprise OTUB1. The linkage may be G76 to K33 and the DUB may comprise at least one of TRABID, or USP21. The linkage may be G76 to K29 and the DUB may comprise at least one of TRABID, or OTUD2. The linkage may be G76 to K27 and the DUB may comprise OTUD2. The linkage may be G76 to K11 and the DUB may comprise at least one of Cezanne, OTUD3, OTUD2, vOTU (virus derived DUB), or USP21; for example the DUB may comprise Cezanne. The linkage may be G76 to K6 and the DUB may comprise at least one of OTUD3, vOTU (virus derived DUB), or USP21. The linkage may be G76 to M1 and the DUB may comprise OTULIN.

In an aspect the invention comprises use of at least one DUB to cleave a conjugate disclosed herein. The conjugate may comprises at least one ubiquitin linkage. The at least one linkage may comprise a G76 to K63 linkage and the at least one DUB may comprise at least one of AMSH, OTUD1, OTUD3, vOTU (virus derived DUB), or USP21; for example the at least one DUB may comprise AMSH and/or OTUD1. The at least one linkage may comprise a G76 to K48 linkage and the at least one DUB may comprise at least one of OTUB1, vOTU (virus derived DUB), USP21, or UCHL3; for example the at least one DUB may comprise OTUB1. The at least one linkage may comprise a G76 to K33 linkage and the at least one DUB may comprise at least one of TRABID, or USP21. The at least one linkage may comprise a G76 to K29 linkage and the at least one DUB may comprise at least one of TRABID, or OTUD2. The at least one linkage may comprise a G76 to K27 linkage and the at least one DUB may comprise OTUD2. The at least one linkage may comprise a G76 to K11 linkage and the at least one DUB may comprise at least one of Cezanne, OTUD3, OTUD2, vOTU (virus derived DUB), or USP21; for example the at least one DUB may comprise Cezanne. The at least one linkage may comprise a G76 to K6 linkage and the at least one DUB may comprise at least one of OTUD3, vOTU (virus derived DUB), or USP21. The at least one linkage may comprise a G76 to M1 linkage and the at least one DUB may comprise OTULIN.

### Formulations and Administration

Conjugates of the invention may be administered topically, intravenously, subcutaneously, buccally, rectally, dermally, nasally, tracheally, bronchially, by any other parenteral route, as an oral or nasal spray or via inhalation. The conjugates may be administered in the form of pharmaceutical preparations comprising prodrug or active conjugate either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical conjugates of the invention may be administered topically, or parenterally (“parenterally” as used herein, refers to modes of administration which include intravenous, intramuscular, intraperitoneal, intrasternal, subcutaneous and intraarticular injection and infusion) to a host to obtain a protease-inhibitory effect. In the case of larger animals, such as humans, the conjugates may be administered alone or as compositions in combination with pharmaceutically acceptable diluents, excipients or carriers.

Actual dosage levels of active ingredients in the pharmaceutical formulations and pharmaceutical compositions of this invention may be varied so as to obtain an amount of the active conjugate(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

According to a further aspect of the invention there is thus provided a pharmaceutical formulation or composition including a conjugate of the invention, optionally in admixture with a pharmaceutically acceptable adjuvant, diluents or carrier.

### Uses

Conjugates of the invention comprising UbiFab and/or UbiMab may be useful in immunotherapy (e.g. radioimmunotherapy). For example, bispecific antibodies are considered to represent promising therapeutics (A. F. Labrijn, et al., PNAS, (2013), 110(13), 5145-5150; S. E. Sedykh, Drug Design, Development and Therapy, (2018), 12, 195-208). Conjugates of the invention (e.g. comprising UbiFab and/or UbiMab) may be used to provide bispecific protein mimetics. These may be useful in the treatment of genetic disorders. For example, bispecific protein mimetics may also be used in therapy. For example, Emicizumab (ACE910) is a protein mimetic comprising a humanized bispecific antibody mimicking the cofactor function of factor VIII, which may be useful in treatment of haemophilia (Sharma, M., et al., N. Engl. J. Med., (2016), 374, 2044-2053; Uchida, N., et al., Blood, (2016), 127(13), 1633-1641).

Conjugates of the present invention may be prepared that are bispecific and approximate antibodies. For example, a conjugate of the invention may comprise two UbiFab moieties, where each UbiFab comprises a Fab with a different specificity. The resulting conjugate provides a bispecific conjugate, which, e.g., may be useful in immunotherapy. In addition, the conjugate may comprise further UbiFab or UbiMab moieties, providing the potential for conjugates with 3, 4, or more specificities. Such conjugates may be used in the treatment of, for example, a cancer, an autoimmune disease, Alzheimer's disease, or a genetic disorder.

The conjugate may also comprise a probe, which may be a payload (e.g. a cytotoxic agent) or a label. Antibody-drug conjugates, which comprise antibody conjugated to a payload are known to have therapeutic potential, e.g. in the treatment of cancer (K. Tsuchikama and Z. An, Protein Cell, (2018), 9(1), 33-46). Conjugates of the present disclosure that comprise UbiFab and/or UbiMab and a payload have similar potential.

Conjugates that comprise a moiety comprising a label and a moiety comprising an affinity species (e.g. a Mab or a Fab) may be used as an in vitro detection tool. For example, the affinity species may target an analyte of interest and the label then permits detection. The label can be directly detectable (fluorophore) or indirectly detectable (hapten or enzyme).

In addition to providing conjugates with tailored specificity (e.g. with one or more moieties comprising Fab or Mab) and optional probes, the use of ubiquitin linkages between the moieties provides further advantages. The methods disclosed herein provide the desired conjugates with a high degree of homogeneity, whereas many conventional methods of forming conjugates provide heterogeneous conjugates. Providing conjugates with a high degree of homogeneity may improve yields. Providing conjugates with a high degree of homogeneity may avoids the need for difficult and/or time consuming separation that would be required to isolate a desired conjugate from the mixture of heterogeneous conjugates. Another advantage of using ubiquitin to link moieties is that ubiquitin is non-immunogenic. Conjugates of the present disclosure should therefore demonstrate reduced and/or low levels of immunogenicity. This may be particularly beneficial when the present conjugates are used in therapy.

In an embodiment a conjugate or formulation of the invention or disclosure is for use as a medicament. The conjugate may comprise at least one (e.g. at least two) UbiFab. The conjugate may comprise at least one UbiMab, optionally where the at least one Mab comprises at least the binding moieties of one of Rituximab, Trastuzumab, Alemtuzumab, Omalizumab, or Emicizumab. The conjugate may comprise a probe, for example a payload (e.g. a cytotoxic agent). The conjugate may comprise at least one (e.g. at least two) UbiFab and a payload (e.g. a cytotoxic agent). The conjugate may comprise at least one UbiMab and a payload (e.g. a cytotoxic agent).

In another embodiment a conjugate or formulation of the invention or disclosure is for use in the treatment of a cancer, an autoimmune disease, Alzheimer's disease, or a genetic disorder. The treatment may be treatment of a cancer. The conjugate may comprise at least one (e.g. at least two) UbiFab. The conjugate may comprise at least one UbiMab, optionally where the at least one Mab comprises at least the binding moieties of Rituximab, Trastuzumab, Alemtuzumab, Omalizumab, or Emicizumab. The conjugate may comprise a probe, for example a payload (e.g. a cytotoxic agent). The conjugate may comprise at least one (e.g. at least two) UbiFab and a payload (e.g. a cytotoxic agent). The conjugate may comprise at least one UbiMab and a payload (e.g. a cytotoxic agent).

Another embodiment provides a method for the treatment of a disease selected from a cancer, an autoimmune disease, Alzheimer's disease, or a genetic disorder, in a patient in need of said treatment by administering an effective amount of a conjugate of the invention or disclosure to the patient. The disease may be a cancer. The conjugate may comprise at least one (e.g. at least two) UbiFab. The conjugate may comprise at least one UbiMab, optionally where the at least one Mab comprises Rituximab, Trastuzumab, Alemtuzumab, Omalizumab, or Emicizumab. The conjugate may comprise a probe, for example a payload (e.g. a cytotoxic agent). The conjugate may comprise at least one (e.g. at least two) UbiFab and a payload (e.g. a cytotoxic agent). The conjugate may comprise at least one UbiMab and a payload (e.g. a cytotoxic agent).

A further embodiment provides a method of performing an assay, the method comprising

(i) contacting a conjugate of the invention with a sample;

(ii) allowing the conjugate associate with an analyte, if said analyte is present; and

(iii) detecting any conjugate associated with said analyte, thereby detecting the analyte;

wherein conjugate comprises a moiety that has affinity for the analyte and the conjugate comprises a label.

The step of contacting may be performed in solution. The step of allowing the conjugate associate with an analyte, if said analyte is present may be performed in solution. The moiety that has affinity for the analyte may comprise at least one (e.g. at least two) Fab portion(s) of UbiFab moiety/moieties of the conjugate. The moiety that has affinity for the analyte may comprise at least one Mab portion of a UbiMab moiety of the conjugate. The label may be directly detectable (e.g. fluorophore) or indirectly detectable (e.g. hapten or enzyme). The label may be a fluorophore, a fluorescent protein, a hapten, or an enzyme.

A further embodiment provides use of a conjugate of the invention in an assay, optionally wherein the assay involves the detection of an analyte. The conjugate may comprise an affinity species that has affinity for the analyte. The conjugate may comprise a label. For example, the conjugate may comprise an affinity species that has affinity for the analyte and a label. The affinity species may be or comprise at least one (e.g. at least two) Fab portion(s) of UbiFab moiety I moieties of the conjugate. The affinity species may be or comprise at least one Mab portion of a UbiMab moiety of the conjugate. The label may be directly detectable (e.g. fluorophore) or indirectly detectable (e.g. hapten or enzyme). The label may be a fluorophore, a fluorescent protein, a hapten, or an enzyme.

### EXAMPLES

### Example 1: Genomic Editing of Hybridoma Cell Lines Using CRISPR/Cas9 to Secrete UbiFabs

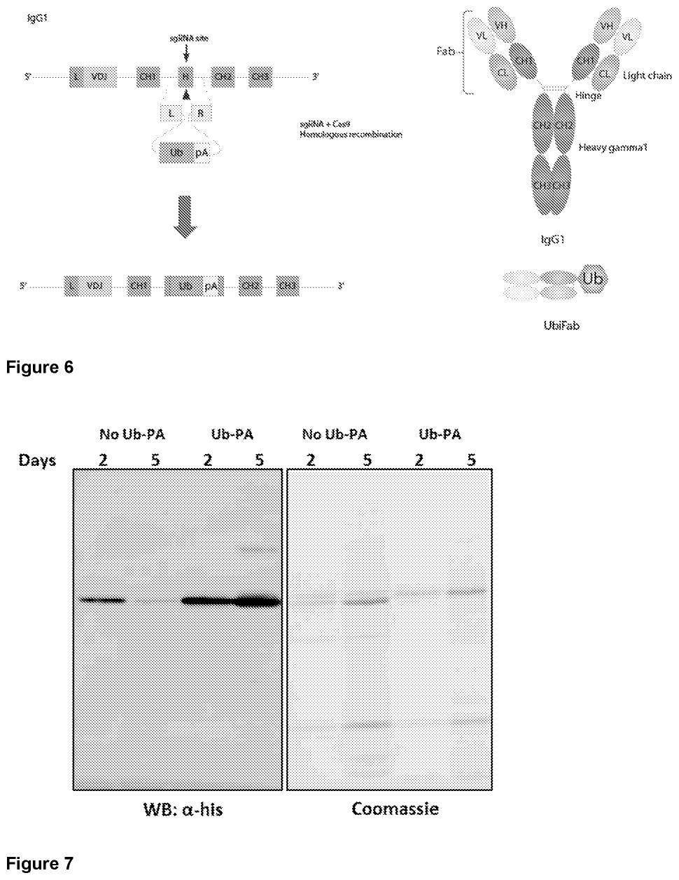

The genome of two hybridoma cell lines, NLDC-145 producing monoclonal antibodies against mouse DEC205 and the cell line OKT-3 producing monoclonal antibodies against mouse CD3, were edited using CRISPR/Cas9 to insert the sequence of ubiquitin mutants followed by a his-tag at the hinge region (FIG. 6). This resulted in stable hybridoma cell lines secreting fab fragments with ubiquitin fused to the C-terminus of the heavy chain. The ubiquitin sequence inserted was either WT ubiquitin, to give called proximal UbiFabs, or with lysine 48 mutated to arginine, forming distal UbiFabs.

An E2/E3 enzyme combination specific for K48 linked ubiquitin was used and therefore contained mutated K48 in the distal UbiFab design.

### Example 2: Expression of UbiFabs

For the production of antibodies, the hybridoma cell lines were cultured for 10 days in serum free hybridoma media supplemented with 2 mM ultraglutamine and 50 μM 2-mercaptoethanol. As shown in FIG. 7, after culturing the hybridoma cells for 5 days, the growth media showed an increase in UbiFabs present in the media, yet there was a decrease in his-tagged UbiFabs compared to day 2. When cultured in media supplemented with 1 μM C-terminally propargylated ubiquitin (Ub-PA), which selectively inhibits DUBS through the reaction with the active site cysteine residue, an increase was observed in time for both his-tagged UbiFabs and the total amount of UbiFabs secreted into the media. This indicates that the C-terminal his-tag is reserved by inhibiting DUBs, which are likely released into the media as cells die during culturing.

The methods for forming conjugates disclosed utilize proximal ubiquitin with a blocked C-terminus and distal ubiquitin with a free C-terminus. A his-tag on a proximal ubiquitin, e.g. forming part of a UbiFabs, can be used for blocking the C-terminal glycine residue of the ubiquitin. Therefore, proximal UbiFabs were secreted in culture media supplemented with Ub-PA. On the other hand, distal UbiFabs typically have a free C-terminus, which can be exposed by DUBs during culturing.

### Example 3: Purification of UbiFabs

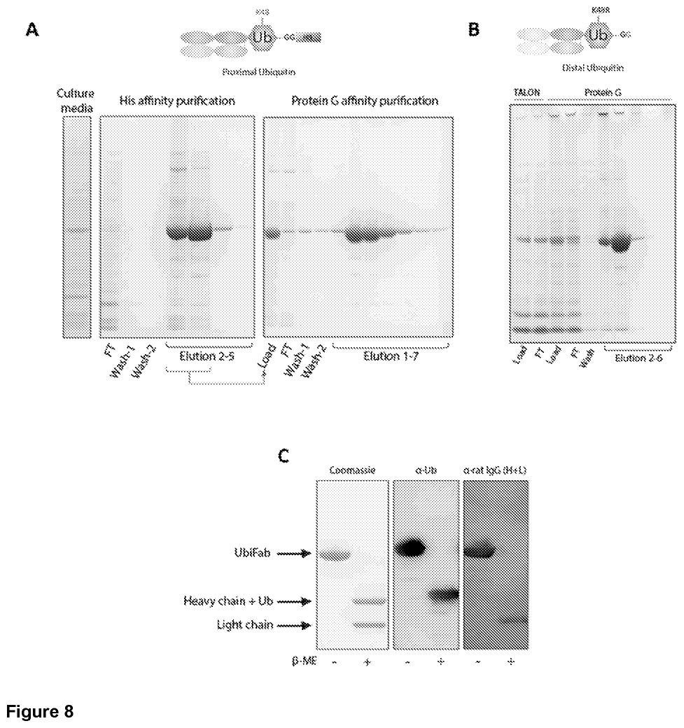

Following the culturing of proximal UbiFab hybridoma cells for 10 days, the culturing media was centrifuged and the supernatant containing proximal UbiFabs and filtered. Next, it was purified using TALON affinity purification, where it was eluted using 250 mM imidazole in 1 mL fractions. The elution fractions containing the proximal UbiFabs were pooled and loaded on a protein G column for further purification (FIG. 8A). Elution fractions from protein G affinity purification were pooled and dialyzed against PBS.

Distal UbiFabs were first passed through a TALON column to retain any distal UbiFabs where the his-tag was not cleaved from the c-terminus during culturing. The flow through was then purified using protein G affinity purification and dialyzed against PBS. Isolation of the distal UbiFabs by this methodology was confirmed by the results indicated in FIG. 8B.

Purified UbiFab showed 2 bands on SDS-PAGE when reduced with β-ME. Western blot analysis indicated that the upper band is the heavy chain with ubiquitin fused to it, while the lower band is the light chain (FIG. 8C).

### Example 4: Antigen-Binding Activity of UbiFabs

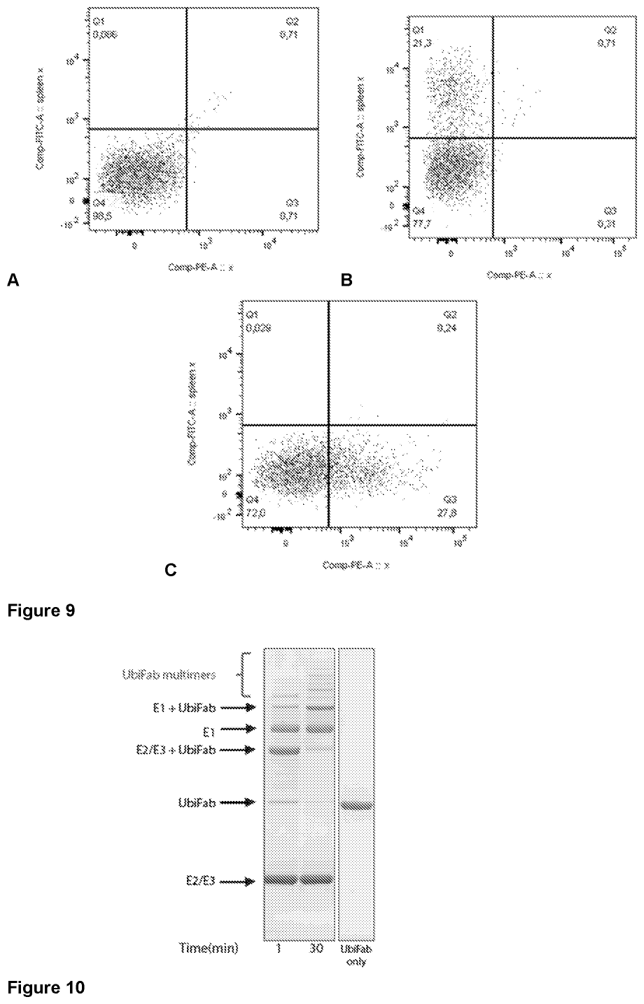

To verify that the antigen-binding activity of anti-CD3 UbiFabs is not adversely affected by fusion to ubiquitin, flowcytometry analysis was carried out using mouse splenocytes. The splenocytes were incubated with UbiFabs for 1 hour followed by washing and incubation with a PE anti-his antibody to visualize the binding. An FITC anti-CD3 monoclonal antibody, of the same clone (OKT3) as the UbiFab, was used as a control. As shown in FIG. 9, cells incubated with UbiFabs (FIG. 9C) showed a comparable percentage of CD3 positive cells to those incubated with anti-CD3 monoclonal antibodies (FIG. 9B). FIG. 9A provides results for the negative (no labeling) control.

### Example 5: Recognition of UbiFabs by Ubiquitinating Enzymes

To investigate whether UbiFabs can be processed by the ubiquitin machinery, UbiFabs with both an exposed C-terminus and lysine residues available for conjugation were incubated with the E1 UBA1, and the K48 linkage specific E2/E3 fusion gp78RING-Ubc7, in presence of ATP and MgCl₂. After 30 minutes, bands of higher molecular weight were formed indicating the formation of UbiFab chains, as illustrated in the gel electrophoresis results shown in FIG. 10.

### Example 6: Site Directed Fluorescent Labelling of UbiFabs by Conjugation to TAMRA-Ubiquitin

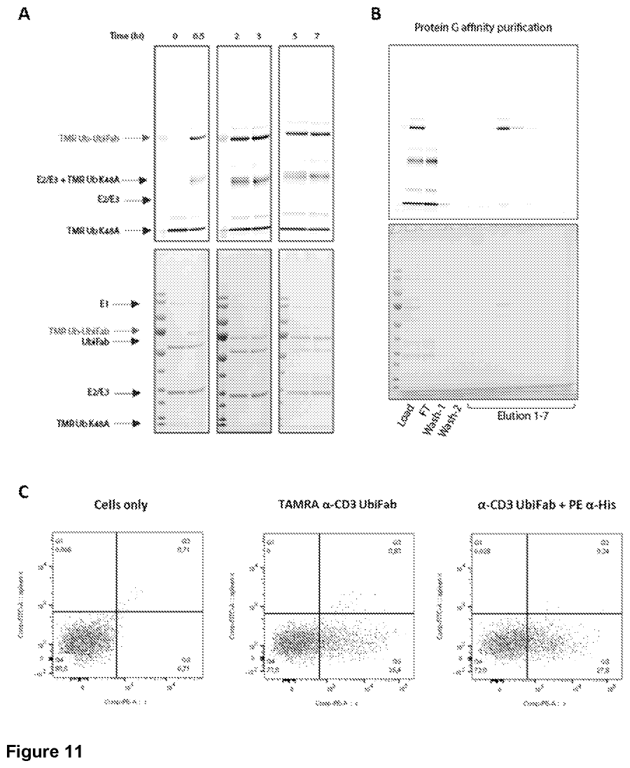

To determine the site specificity of UbiFab conjugation, anti-CD3 proximal UbiFabs were enzymatically conjugated to the synthetic TAMRA-labelled ubiquitin mutant K48A (TMR-UbK48A) using the same conditions mentioned previously. After 30 mins, a fluorescent band appeared around 66 kDa, which in time increased in intensity (FIG. 11A). No other bands appeared, indicating the site specificity of the reaction at lysine 48. Conjugated UbiFabs were isolated from the reaction mixture using protein G affinity purification (FIG. 11B).

Flow cytometry analysis of mouse splenocytes stained with the TMR-UbK48A conjugated anti CD3 UbiFab showed a comparable percentage of CD3 positive population compared to unconjugated anti-CD3 UbiFabs visualized using PE anti-his antibody (FIG. 11C).

### Example 7: UbiFabs are Site Specifically Conjugated to Form Bi-Specific UbiFabs

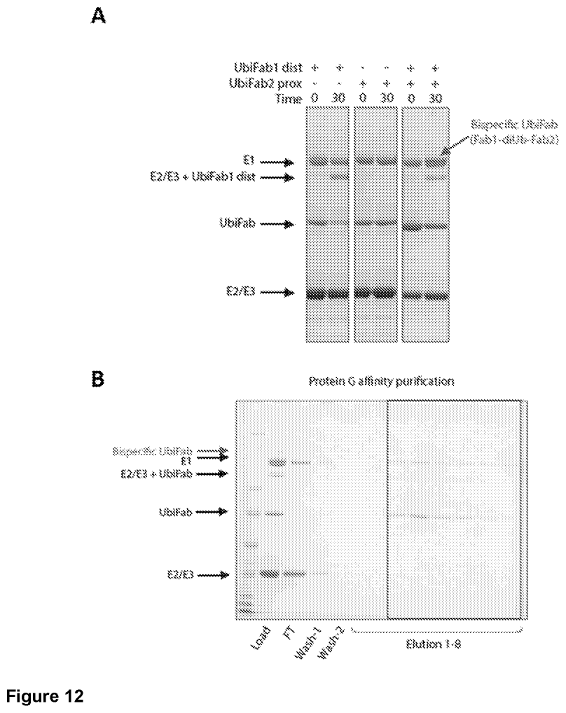

To site specifically conjugate two different UbiFabs forming a bi-specific UbiFab, anti CD3 proximal UbiFab was conjugated to anti DEC205 distal UbiFab. For this, the same reaction conditions were used as mentioned in earlier Examples. As a control, each of proximal UbiFabs and distal UbiFabs were reacted in absence of the other. As shown in FIG. 12A, after 30 minutes a band appeared only in the reaction mixture where both proximal and distal UbiFabs were present indicating site specific conjugation at K48. An additional band can be observed after 30 minutes in both the sample containing only distal UbiFabs and the sample containing both proximal and distal UbiFabs. This band corresponds to the E2/E3 enzymes covalently bound to the C-terminus of distal UbiFabs.

The formed bi-specific UbiFabs were then purified using Protein G affinity purification (FIG. 12B) followed by gel filtration to remove unreacted UbiFabs.

### Example 8: Exposure of the C-Terminus of UbiFabs Through the Cleavage by a Deubiquitinating Enzyme (DUB)

Removing the C-terminal his-tag from the C-terminus of UbiFabs will expose the C-terminal glycine residue of ubiquitin required for ubiquitination. To determine if UbiFabs are recognized by and the his-tag cleaved using a DUB, 40 μM anti-CD3 proximal UbiFabs were incubated with 50 nM UCHL3, a DUB known to cleave short peptides off the C-terminus of ubiquitin, for 30 minutes at room temperature. To follow the reaction at different time points, samples were reduced using 10 mM TCEP for 10 minutes and measured on LC-MS. The deconvoluted mass spectrum at the top of FIG. 13 shows two peaks corresponding to the light chain and heavy chain/ubiquitin fusion of anti-CD3 proximal UbiFab. The mass of the heavy chain/ubiquitin fusion of 33,863 Da corresponds to the presence of a 10× his-tag. After 30 mins the mass of the heavy chain/ubiquitin fusion is reduced by 1372 Da, as indicated in the deconvoluted mass spectrum at the bottom of FIG. 13. This indicates that the his-tag is cleaved. The exposed C-terminal glycine residue is then available for ubiquitination.

### Example 9: Multimerization of UbiFabs

Multimerization of UbiFabs was demonstrated using the E2 UbcH7 and E3 NIeL for the assembly of both K6- and K48-linked ubiquitin chains. In these experiments, the UbiFabs that were used had all of their lysine residues and their C-termini available for conjugation. After 3 hours under reaction conditions as described in example 5, the reaction products were separated using gel electrophoresis. As illustrated in FIG. 14, coomassie staining showed bands of high molecular weight which increased in intensity upon further incubation in presence of additional E2 and E3 enzymes. These bands correspond to the formation of UbiFab multimers linked at K6 and/or K48.

### Example 10: Disassembly of UbiFabs

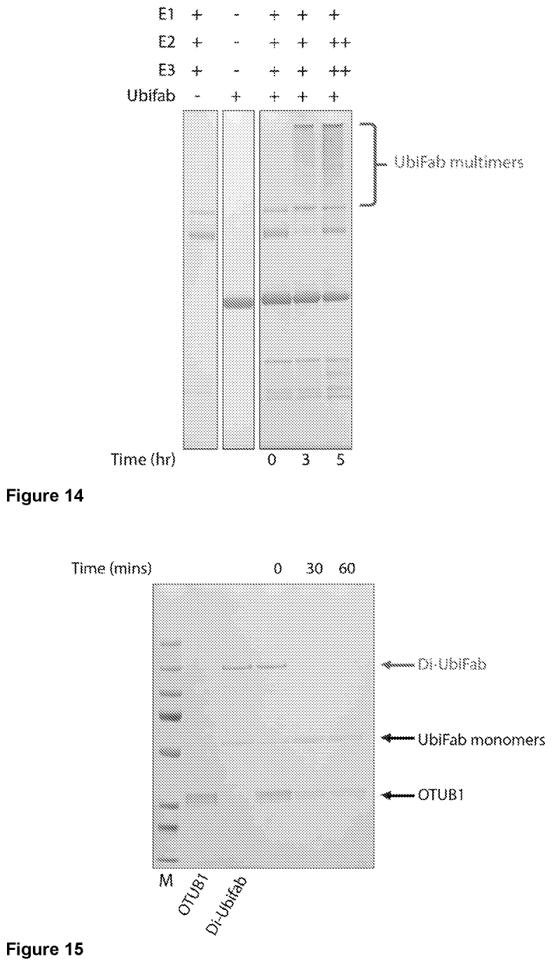

Disassembly of a UbiFab conjugate has been performed with a DUB. In this example OTUB1, a K48-specific DUB was used. The UbiFab conjugate was a di-UbiFab with a ubiquitin linkage from G76 of the ubiquitin of the distal UbiFab to K48 of the ubiquitin of the proximal UbiFab. The UbiFab and OTUB1 were contacted in buffer solution. Samples were obtained at time points of 30 and 60 minutes. A sample was also obtained prior to commencement of the reaction to provide time point 0. The samples were analysed by gel electrophoresis, with the results obtained indicated in FIG. 15. After 30 minutes, the band corresponding to the di-ubiFab disappears while the band corresponding to ubiFab monomers increase in intensity. This indicates that ubiFabs can be readily cleaved using DUBs.

### Example 11: UbiFab Trimer

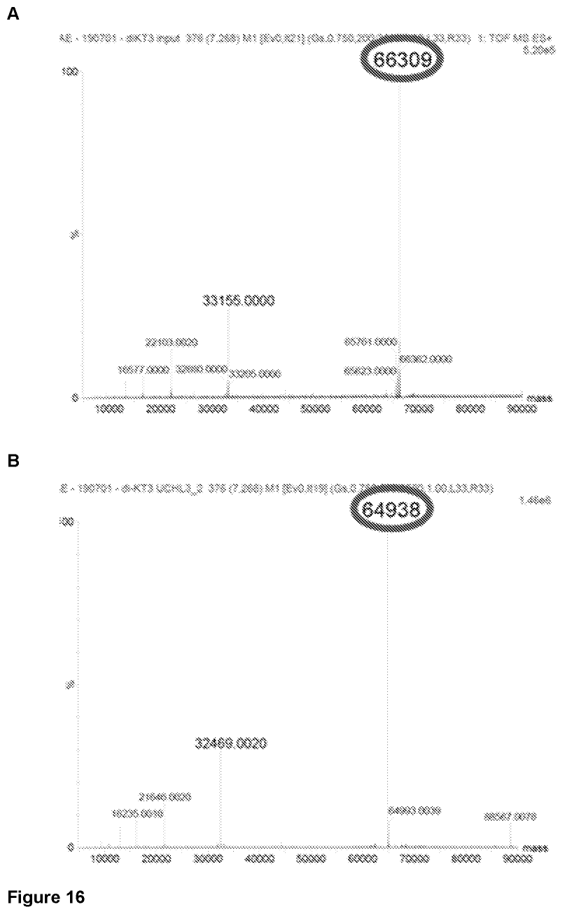

To further elongate the UbiFab heterodimer by conjugating a third moiety, it is required to expose the C-terminal glycine residue of the proximal UbiFab. This was efficiently done using the deubiquitinating enzyme UCHL3, where the reaction was followed by LC-MS. After 30 minutes, 100% cleavage of the histidine-tag and exposure of the C-terminal glycine was observed, as illustrated by the results in FIG. 16. The cleaved dimer was easily separated from the reaction mixture using 50 kDa cut-off spin filtration.

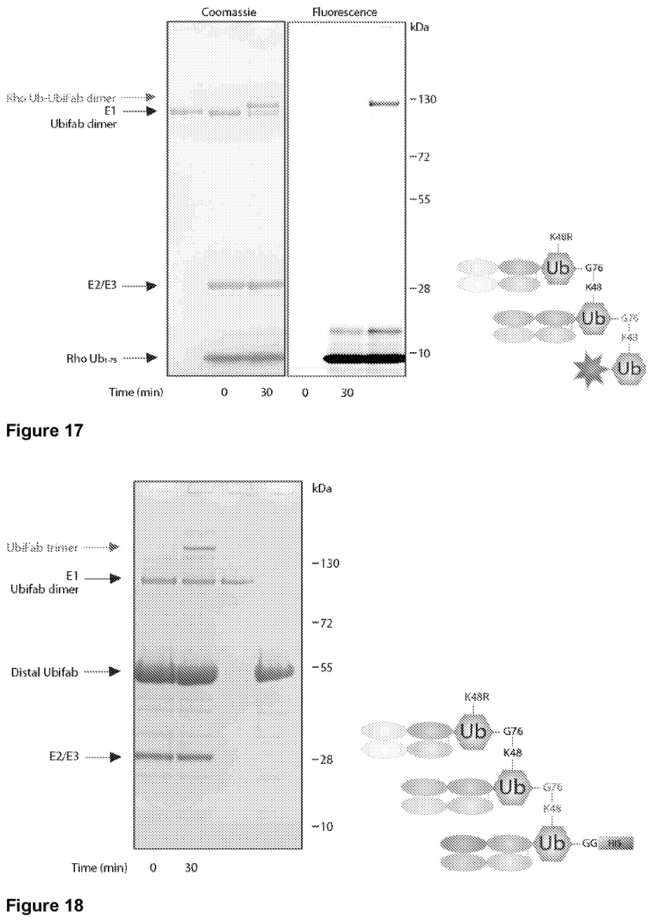

The purified dimer having a free C-terminal glycine was used to conjugate either a third UbiFab or a chemically synthesized Rhodamine-Ubiquitin, forming a hetero-trimer or a rhodamine-labelled UbiFab-dimer respectively. For this, the third moiety requires the lysine-48 to be available for conjugation while the C-terminal glycine is blocked. Accordingly, either a post-proximal UbiFab (e.g., see FIG. 5B) or Rho-Ub75 were used. Rho-Ub75 represents a synthetic ubiquitin with a Rhodamine moiety attached to the ubiquitin N-terminus and the C-terminus blocked by the omission of Gly-76. The results provided in FIG. 17 confirm the formation of a trimer comprising UbiFab heterodimer conjugated with Rhodamine-Ubiquitin, with incorporation of the Rhodamine dye confirmed by fluorescence. The results in FIG. 18 illustrates the formation of a trimer where a post-proximal UbiFab was reacted with the C-terminal glycine of the proximal UbiFab of the heterodimer.

### Example 12: Thermal Unfolding (Stability)

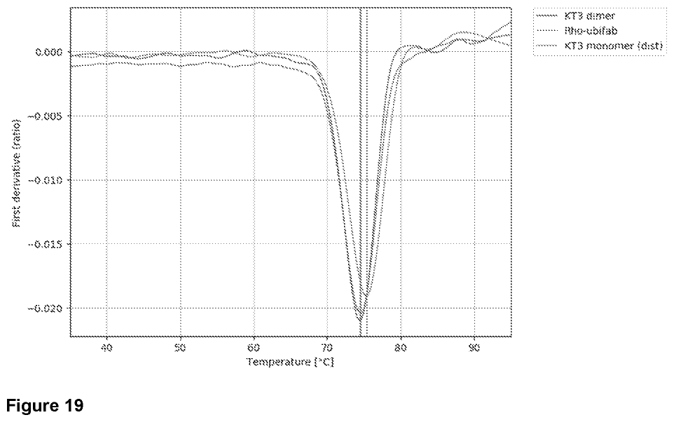

Thermal stability was investigated and confrimed that the ubiquitin linked conjugates retain their protein stability after conjugation. This was determined by comparing the thermal unfolding of conjugated ubifab dimers and flourescently labelled ubifabs to the parent ubifab monomers. The species tested were: an anti-CD3(mouse) distal UbiFab (monomer); a UbiFab dimer consisting of a proximal and a distal anti-CD3 UbiFab moieties (different type of UbiFabs having the same target); and a distal anti-CD3 UbiFab conjugated to Rho-Ub75. As illustrated by the results presented in FIG. 19, the conjugated ubifabs showed comparable thermostability to the parent ubifabs, indicating that the ubiquitin-based conjugation does not compromise the protein stability.

### Example 13: UbiFab Conjugation to Ubiquitin-Peptide

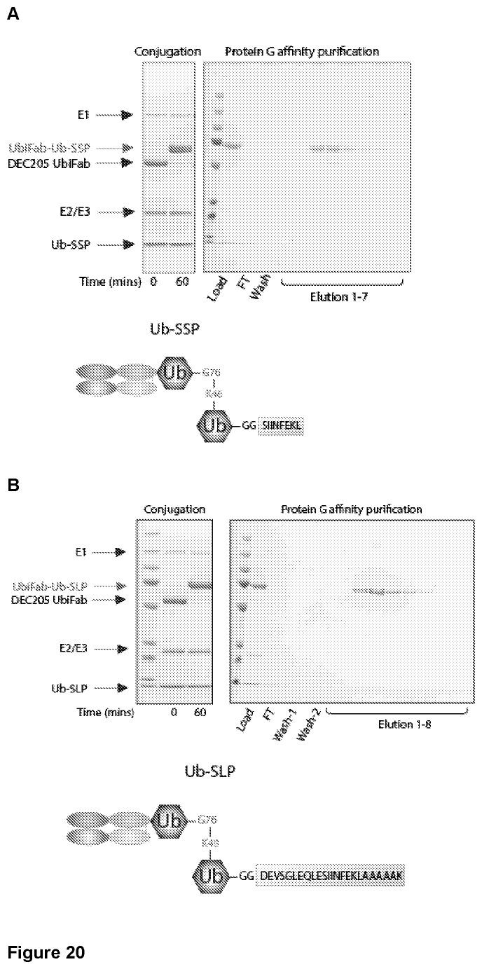

Oligo and polypeptides were conjugated to an antibody fragment using ubiquitin conjugation. This example used an anti-DEC205 distal UbiFab and a chemically synthetized ubiquitin of which the C-terminus is followed by an ovalbumin-derived oligo- or poly-peptide, Ubi-SSP or Ubi-SLP respectively. SSP has the sequence SIINFEKL (SEQ ID NO. 4) and SLP has the sequence DEVSGLEQLESIINFEKLAAAAAK (SEQ ID NO. 5). Both conjugation reactions involving either Ubi-SSP or Ubi-SLP were highly efficient, with an approximate yield of 80-90%. FIG. 20A illustrates the results obtained for the conjugation of anti-DEC205 distal UbiFab with proximal Ubi-SSP. FIG. 20B illustrates the results obtained for the conjugation of anti-DEC205 distal UbiFab with proximal Ubi-SLP.

### Example 14: MHC-I Ubi-Multimers

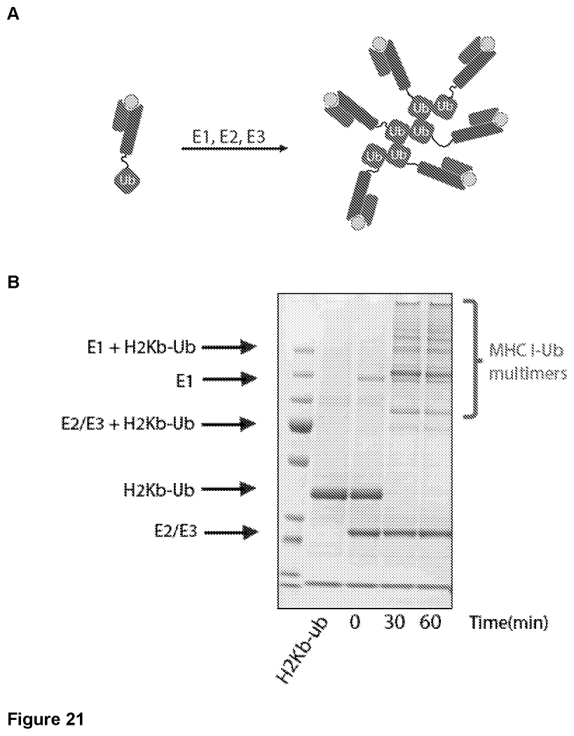

The applicability of ubiquitin based conjugation of proteins beyond the scope of antibody conjugation was demonstrated with ubiquitin-based multimerization of MHC class I. The monomeric moieties used were MHC-I H-2Kb with ubiquitin fused to the C-terminus of the heavy chain (SEQ ID NO. 6). The fused ubiquitin has both the lysine residue (K48) as well as the C-terminus available for conjugation, resulting in the formation of ubiquitin-linked MHC-I multimers. After 60 minutes complete conversion of MHC-I monomers to multimers of various chain length was observed. The overall reaction is illustrated in FIG. 21A, while FIG. 21B provides a coomassie stained gel, confirming the formation of MHC-I Ubi-multimers.

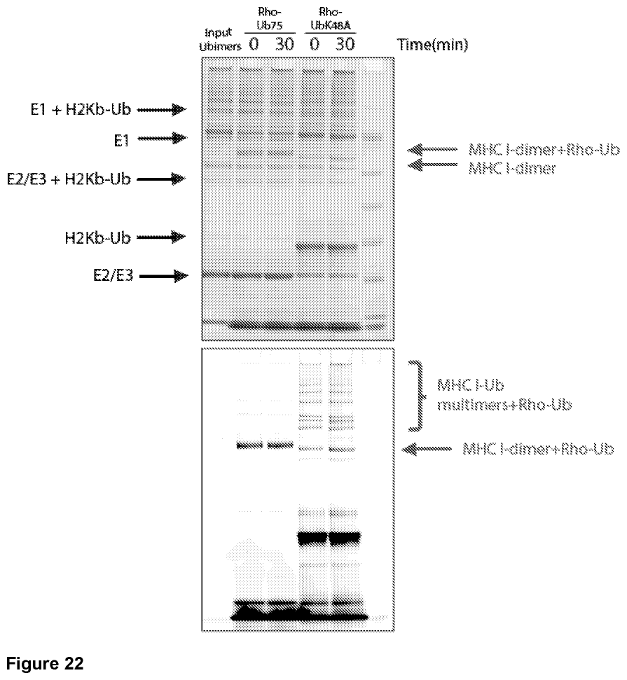

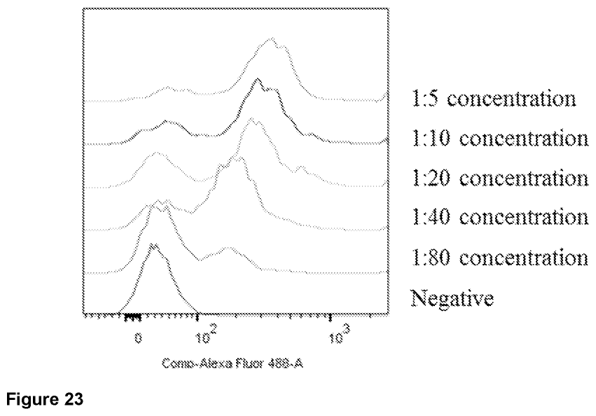

The MHC-I multimers were fluorescently labelled. Rho-labelled ubiquitin was added in excess to the reaction mixture, in the form of either Rho-Ub75 or Rho-UbK48A. Rho-Ub75 is a synthetic ubiquitin with a Rhodamine moiety attached to the ubiquitin N-terminus and the C-terminus blocked by the omission of Gly-76. Rho-UbK48A is a synthetic ubiquitin K48A with a Rhodamine moiety attached to the ubiquitin N-terminus. As indicated by the fluorescence results (lower image) of FIG. 22, the reaction involving Rho-UbK48A was more efficient. The functionality of the formed Rhodamine labelled MHC-I multimers was validated using flowcytometry (FIG. 23); where OT-I CD8+ T cells were efficiently stained using the ubiquitin-linked MHC I multimers.

### Sequences

Information on sequences referenced herein that are disclosed in public databases is provided in the table 4:

TABLE 4
Sequences

| Description | Database reference |
|---|---|
| Ubiquitin | P0CG48 (UBC_HUMAN); P0CG47 |
| | (UBB_HUMAN) |
| E1 | P22314 (UBA1_HUMAN) |
| E2 enzyme | Q9UKV5 (AMFR_HUMAN) |
| (for ubiquitin K 48) | |
| E3 enzyme | P60604 (UB2G2_HUMAN) |
| (for ubiquitin K48) | |
| Rat gamma-2a | Genomic: |
| immunoglobulin | M13804.1, AAA41376.1; M28669.1, |
| heavy chain | AAA60737.1 |
| | mRNA: |
| | BC088240.1, AAH88240.1 |
| DUB Enzymes | |
| AMSH | O95630 (STABP_HUMAN) |
| Cezanne | Q6GQQ9 (OTU7B_HUMAN) |
| OTUB1 | Q96FW1 (OTUB1_HUMAN) |
| OTUD1 | Q5VV17 (OTUD1_HUMAN) |
| OTUD3 | Q5T2D3 (OTUD3_HUMAN) |
| TRABID | Q9UGI0 (ZRAN1_HUMAN) |
| OTUD2 | Q5VVQ6 (OTU1_HUMAN) |
| USP21 | Q9UK80 (UBP21_HUMAN) |
| OTULIN | Q96BN8 (OTUL_HUMAN) |
| TRABID | Q9UGI0 (ZRAN1_HUMAN) |
| UCHL3 | P15374 (UCHL3_HUMAN) |

The following sequences are also provided:

Ubiquitin, amino acids 1 to 76 of UBB_HUMAN
SEQ ID NO: 1
MQIFVKTLTGKTITLEVEPSDTIENVKAKIQDKEGIPPDQQRLIFAGKQLE
DGRTLSDYNIQKESTLHLVLRLRGG
Ubc7-gp78RING fusion protein based on Ubc7 of
AMFR_HUMAN and gp78RING domain of UB2G2_HUMAN
SEQ ID NO: 2
EARFAVATPEELAVNNDDCAICWDSMQAARKLPCGHLFHNSCLRSWLEQDT
SCPTCRMSLNIADNNRVREEGTGSHMNEENFFEWEALIMGPEDTCFEFGVF
PAILSFPLDYPLSPPKMRFTCEMFHPNIYPDGRVCISILHAPGDDPMGYES
SAERWSPVQSVEKILLSVVSMLAEPNDESGANVDASKMWRDDREQFYKIAK
QIVQKSLGL
Peptide SSP, amino acids 258-265 of OVAL_CHICK
SEQ ID NO: 4
SIINFEKL
Peptide SLP, amino acids 248-265 of OVAL_CHICK and
A5K
SEQ ID NO: 5
DEVSGLEQLESIINFEKLAAAAAK
fusion protein based on MHC-I H-2Kb and UBB_HUMAN
SEQ ID NO: 6
MMGPHSLRYFVTAVSRPGLGEPRYMEVGYVDDTEFVRFDSDAENPRYEPRA
RWMEQEGPEYWERETQKAKGNEQSFRVDLRTLLGYYNQSKGGSHTIQVISG
CEVGSDGRLLRGYQQYAYDGCDYIALNEDLKTWTAADMAALITKHKWEQAG
EAERLRAYLEGTCVEWLRRYLKNGNATLLRTDSPKAHVTHHSRPEDKVTLR
CWALGFYPADITLTWQLNGEELIQDMELVETRPAGDGTFQKWASVVVPLGK
EQYYTCHVYHQGLPEPLTLRWEPPGSGGSGGSAGGMQIFVKTLTGKTITLE
VEPSDTIENVKAKIQDKEGIPPDQQRLIFAGKQLEDGRTLSDYNIQKESTL
HLVLRLRGG

1. A conjugate that comprises a ubiquitin dimer or multimer, comprising:
a distal moiety conjugated to a proximal moiety;
wherein the distal moiety comprises a polypeptide comprising a distal ubiquitin at its C-terminus, said ubiquitin comprising at least one of the following mutations: K6X, K11X, K27X, K29X, K33X, K48X, or K63X, where X is selected from R, A or C;
wherein the proximal moiety comprises a polypeptide comprising either a proximal ubiquitin at its C-terminus, or a proximal ubiquitin at its N-terminus;
wherein the distal moiety is conjugated to the proximal moiety via an amide bond from G76 of the distal ubiquitin to one of M1, K6, K11, K27, K29, K33, K48, or K63 of the proximal ubiquitin.

2. The conjugate of claim 1, wherein the proximal moiety comprises a polypeptide comprising a proximal ubiquitin at its C-terminus, said ubiquitin optionally comprising a blocked C-terminus.

3. The conjugate of claim 1, wherein the distal moiety and/or proximal moiety comprise a fusion protein with ubiquitin at its C-terminus.

4. The conjugate of claim 1, wherein the distal moiety comprises a fusion protein comprising a biologically and/or pharmaceutically active polypeptide or peptide and the distal ubiquitin at its C-terminus.

5. The conjugate of claim 1, wherein the distal moiety comprises a fusion protein comprising an antigen binding antibody fragment and the distal ubiquitin at its C-terminus (UbiFab), or a fusion protein comprising a monoclonal antibody and the distal ubiquitin at the C-terminus of the heavy chain or the light chain of the monoclonal antibody (UbiMab).

6. The conjugate of claim 1, wherein the proximal moiety comprises a fusion protein comprising a biologically and/or pharmaceutically active polypeptide or peptide and the proximal ubiquitin at its C-terminus.

7. The conjugate of claim 1, wherein the proximal moiety comprises a fusion protein comprising an antigen binding antibody fragment and the proximal ubiquitin at its C-terminus (UbiFab), or a fusion protein comprising a monoclonal antibody and the proximal ubiquitin at the C-terminus of the heavy chain or the light chain of the monoclonal antibody (UbiMab).

8. The conjugate of claim 1, wherein the distal moiety is a UbiFab and the proximal moiety is a UbiFab.

9. The conjugate of claim 1, wherein at least one of the distal ubiquitin and proximal ubiquitin is substituted with a probe.

10. The conjugate of claim 9, wherein the probe is a label, optionally a fluorophore.

11. The conjugate of claim 1, wherein the distal ubiquitin comprises at least one of the following mutations: K6R, K11R, K27R, K29R, K33R, K48R, K48C, K63R, or K63C.

12. The conjugate of claim 1, wherein the distal moiety is conjugated to the proximal moiety via an amide bond from G76 of the distal ubiquitin to one of K6, K11, K29, K33, K48, or K63 of the proximal ubiquitin.

13. The conjugate of claim 1, wherein the distal moiety is conjugated to the proximal moiety via an amide bond from G76 of the distal ubiquitin to K48 or K63 of the proximal ubiquitin.

14. The conjugate of claim 1, wherein the distal moiety is conjugated to the proximal moiety via an amide bond from G76 of the distal ubiquitin to K48 of the proximal ubiquitin.

15. The conjugate of claim 1, wherein said ubiquitin comprising a blocked C-terminus comprises a deleted G76, or G76-Z where -Z is a sequence of one or more amino acids.

16. The conjugate of claim 15, wherein the blocked C-terminus comprises G76-Z.

17. The conjugate of claim 15, wherein -Z is a His-tag.

18. The conjugate of claim 1, wherein the non-ubiquitin portion of the distal moiety and the non-ubiquitin portion of the proximal moiety differ, thereby providing a bifunctional conjugate.

19. The conjugate of claim 1, further comprising a pre-distal moiety conjugated to the distal moiety;
wherein the pre-distal moiety comprises a pre-distal ubiquitin at its C-terminus, said ubiquitin comprising at least one of the following mutations: K6X, K11X, K27X, K29X, K33X, K48X, or K63X, where X is selected from R, A or C;
wherein the pre-distal moiety is conjugated to the distal moiety via an amide bond from G76 of the pre-distal ubiquitin to one of K6, K11, K27, K29, K33, K48, or K63 of the distal ubiquitin.

20. The conjugate of claim 19, further comprising a pre-pre-distal moiety conjugated to the pre-distal moiety;
wherein the pre-pre-distal moiety comprises a pre-pre-distal ubiquitin at its C-terminus, said ubiquitin comprising at least one of the following mutations: K6X, K11X, K27X, K29X, K33X, K48X, or K63X, where X is selected from R, A or C;
wherein the pre-pre-distal moiety is conjugated to the pre-distal moiety via an amide bond from G76 of the pre-pre-distal ubiquitin to one of K6, K11, K27, K29, K33, K48, or K63 of the pre-distal ubiquitin.

21. The conjugate of claim 19, wherein at least three of the pre-pre-distal moiety (when present), pre-distal moiety, distal moiety and proximal moiety comprises a fusion protein comprising a biologically and/or pharmaceutically active polypeptide or peptide and said ubiquitin at its C-terminus; optionally wherein each said biologically and/or pharmaceutically active polypeptide or peptide is a major histocompatibility complex (MHC), optionally an MHC class I.

22. A conjugate that comprises a ubiquitin multimer, comprising:
a most distal monomeric moiety conjugated to a most proximal monomeric moiety via n intermediate monomeric moieties;
wherein each monomeric moiety comprises a polypeptide comprising a ubiquitin at its C-terminus;
wherein the most distal monomeric moiety conjugated to a first intermediate monomeric moiety via an amide bond from G76 of the most distal monomeric moiety's ubiquitin to one of K6, K11, K27, K29, K33, K48, or K63 of the first intermediate monomeric moiety's ubiquitin;
wherein the n^{th} intermediate monomeric moiety is conjugated the most proximal monomeric moiety via an amide bond from G76 of the n^{th} intermediate monomeric moiety's ubiquitin to one of K6, K11, K27, K29, K33, K48, or K63 of the most proximal monomeric moiety's ubiquitin,
wherein n is an integer from 1 to 100.

23. The conjugate of claim 22, wherein the conjugate comprises each of the n intermediate monomers as follows:
the immediately distal monomeric monomer is conjugated to the x^{th} intermediate monomeric moiety via an amide bond from G76 of the immediately distal monomeric moiety's ubiquitin to one of K6, K11, K27, K29, K33, K48, or K63 of the x^{th} intermediate monomeric moiety's ubiquitin; and
the x^{th} intermediate monomeric moiety is conjugated the immediately proximal monomeric moiety via an amide bond from G76 of the x^{th} intermediate monomeric moiety's ubiquitin to one of K6, K11, K27, K29, K33, K48, or K63 of the immediately proximal monomeric moiety's ubiquitin;
wherein the immediately distal monomeric moiety is the most distal moiety when the x^{th} intermediate monomeric moiety is the first intermediate monomeric moiety, or the immediately distal monomeric moiety is the (x-1)^{th} intermediate monomeric moiety when the x^{th} intermediate monomeric moiety is any intermediate monomeric moiety other than the first intermediate monomeric moiety; and
wherein the immediately proximal monomeric moiety is the most proximal moiety when the x^{th} intermediate monomeric moiety is the n^{th} intermediate monomeric moiety, or the immediately proximal monomeric moiety is the (x+1)^{th} intermediate monomeric moiety when the x^{th} intermediate monomeric moiety is any intermediate monomeric moiety other than the n^{th} intermediate monomeric moiety.

24. The conjugate of claim 22, wherein each monomeric moiety, other than the most proximal monomeric moiety, is conjugated to its immediately proximal moiety via an amide bond from G76 of each monomeric moiety's distal ubiquitin to one of K6, K11, K29, K33, K48, or K63 of its immediately proximal moiety's ubiquitin.

25. The conjugate of claim 22, wherein each monomeric moiety, other than the most proximal monomeric moiety, is conjugated to its immediately proximal moiety via an amide bond from G76 of each monomeric moiety's distal ubiquitin to K48 or K63 of its immediately proximal moiety's ubiquitin.

26. The conjugate of claim 22, wherein each monomeric moiety, other than the most proximal monomeric moiety, is conjugated to its immediately proximal moiety via an amide bond from G76 of each monomeric moiety's distal ubiquitin to K48 of its immediately proximal moiety's ubiquitin.

27. The conjugate of claim 22, wherein the or each intermediate moiety comprises a fusion protein with ubiquitin at its C-terminus;
optionally wherein the most distal monomeric moiety comprises a fusion protein with ubiquitin at its C-terminus; and/or
optionally wherein the most proximal monomeric moiety comprises a fusion protein with ubiquitin at its C-terminus.

28. The conjugate of claim 22, wherein the or each intermediate moiety comprises a fusion protein comprising a biologically and/or pharmaceutically active polypeptide or peptide and the ubiquitin at its C-terminus;
optionally wherein the most distal monomeric moiety comprises a fusion protein comprising a biologically and/or pharmaceutically active polypeptide or peptide and the ubiquitin at its C-terminus; and/or
optionally wherein the most proximal monomeric moiety comprises a fusion protein comprising a biologically and/or pharmaceutically active polypeptide or peptide and the ubiquitin at its C-terminus.

29. The conjugate of claim 28, wherein the or each biologically and/or pharmaceutically active polypeptide or peptide is a major histocompatibility complex (MHC) polypeptide, an antigen binding antibody fragment (Fab) or a monoclonal antibody (Mab).

30. The conjugate of claim 28, wherein the or each biologically and/or pharmaceutically active polypeptide or peptide is a major histocompatibility complex (MHC) polypeptide.

31. The conjugate of claim 22, wherein at least one of the monomeric moieties comprises a ubiquitin substituted with a probe.

32. The conjugate of claim 22, wherein the most distal monomeric moiety comprises a ubiquitin substituted with a probe and/or the most proximal monomeric moiety comprises a ubiquitin substituted with a probe.

33. The conjugate of claim 31, wherein the probe is a label, optionally a fluorophore.

34. A method for the production of a conjugate, comprising:
(i) providing a solution comprising a distal moiety, a proximal moiety, a ubiquitin activating enzyme (E1), a ubiquitin-conjugating enzyme (E2) and optionally a ubiquitin-ligating enzyme (E3);
wherein the distal moiety comprises a distal ubiquitin at its C-terminus, the distal ubiquitin optionally comprising at least one of the following mutations: K6X, K11X, K27X, K29X, K33X, K48X, or K63X, where X is selected from R, A or C;
wherein the proximal moiety comprises a polypeptide comprising a proximal ubiquitin at its C-terminus, or a proximal ubiquitin at it N-terminus, said ubiquitin comprising a blocked C-terminus; and
(ii) thereby forming a first conjugate such that the distal moiety is conjugated to the proximal moiety via an amide bond from G76 of the distal ubiquitin to one of M1, K6, K11, K27, K29, K33, K48, or K63 of the proximal ubiquitin.

35. The method of claim 34, wherein the solution comprises a distal moiety, a proximal moiety, a ubiquitin activating enzyme (E1), a ubiquitin-conjugating enzyme (E2) and a ubiquitin-ligating enzyme (E3).

36. The method of claim 34, wherein the distal moiety is conjugated to the proximal moiety via an amide bond from G76 of the distal ubiquitin to K48 or K63 of the proximal ubiquitin.

37. The method of claim 34, wherein the distal ubiquitin comprises the mutation K48R or K48C and the amide bond is from G76 of the distal ubiquitin to K48 of the proximal ubiquitin.

38. The method of claim 34, wherein the E2 and E3 enzymes are Mms2 and Ubc13, or Ubc13 and Uev1A, or UbcH7 and Gp78, or UbcH7 and NleL.

39. The method of claim 34, wherein the blocked C-terminus comprises a deleted G76, or G76-Z where —Z is a sequence of one or more amino acids.

40. The method of claim 39, wherein the blocked C-terminus comprises G76-Z.

41. The method of claim 40, wherein -Z is a His-tag.

42. The method of claim 34, wherein the distal moiety comprises a distal fusion protein with the distal ubiquitin at its C-terminus; and/or
wherein the proximal moiety comprises a proximal fusion protein with the proximal ubiquitin at its C-terminus.

43. The method of claim 42, wherein the distal fusion protein comprises a biologically and/or pharmaceutically active polypeptide or peptide and the distal ubiquitin at its C-terminus.

44. The method of claim 42, wherein the proximal fusion protein comprises a biologically and/or pharmaceutically active polypeptide or peptide and the proximal ubiquitin at its C-terminus.

45. The method of claim 42, wherein the distal fusion protein comprises an antigen binding antibody fragment and the distal ubiquitin at its C-terminus (UbiFab), or the distal fusion protein comprises a monoclonal antibody and the distal ubiquitin at the C-terminus the heavy chain or the light chain of the monoclonal antibody (UbiMab).

46. The method of claim 42, wherein the proximal fusion protein comprises an antigen binding antibody fragment and the proximal ubiquitin at its C-terminus (UbiFab), or the proximal fusion protein comprises a monoclonal antibody and the proximal ubiquitin at the C-terminus the heavy chain of the monoclonal antibody (UbiMab).

47. The method of claim 42, wherein providing the distal moiety comprises expressing the distal fusion protein in a cell and isolating the distal fusion protein, optionally wherein the cell is a eukaryotic cell.

48. The method of claim 42, wherein providing the proximal moiety comprises expressing the proximal fusion protein in a cell and isolating the proximal fusion protein, optionally wherein the cell is a eukaryotic cell.

49. The method of claim 48, wherein the blocked C-terminus comprises G76-His-tag and expressing the proximal fusion protein comprises culturing said eukaryotic cell in a medium supplemented with a deubiquitinating enzyme (DUBs) inhibitor, optionally wherein the DUBs inhibitor is or comprises propargylated ubiquitin (Ub-PA).

50. The method of claim 47, wherein each eukaryotic cell is a CHO cell or a hybridoma cell.

51. The method of claim 47, further comprising:
(iii) unblocking the blocked C-terminus of the first conjugate to provide an unblocked first conjugate.

52. The method of claim 51, wherein the blocked C-terminus comprises G76-His-tag and the unblocking comprises contacting the conjugate with a deubiquitinating enzyme.

53. The method of claim 51, further comprising:
(iv) providing a solution comprising the unblocked first conjugate, a post-proximal moiety, a ubiquitin activating enzyme (E1), a ubiquitin-conjugating enzyme (E2) and a optionally a ubiquitin-ligating enzyme (E3);
wherein the post-proximal moiety comprises a polypeptide comprising a post-proximal ubiquitin at its C-terminus, the post-proximal ubiquitin comprising a blocked C-terminus, or a post-proximal ubiquitin at its N-terminus; and
(v) thereby forming a second conjugate such that the unblocked first conjugate is conjugated to the post-proximal moiety via an amide bond from G76 of the proximal ubiquitin to one of M1, K6, K11, K27, K29, K33, K48, or K63 of the post-proximal ubiquitin.

54. The method of claim 34, further comprising:
(vi) providing a solution comprising a pre-distal moiety, the first conjugate or the second conjugate, a ubiquitin activating enzyme (E1), a ubiquitin-conjugating enzyme (E2) and optionally a ubiquitin-ligating enzyme (E3);
wherein the pre-distal moiety comprises a pre-distal ubiquitin at its C-terminus, the distal ubiquitin comprising at least one of the following mutations: K6X, K11X, K27X, K29X, K33X, K48X, or K63X, where X is selected from R, A or C; and
(vii) thereby forming a third conjugate such that the pre-distal moiety is conjugated to the first conjugate or the second conjugate via an amide bond from the G76 of the pre-distal ubiquitin to one of K6, K11, K27, K29, K33, K48, or K63 of the distal ubiquitin or the proximal ubiquitin, or (if present) the post-proximal ubiquitin.

55. A method for the production of a multimeric conjugate, comprising:
(i) providing a solution comprising a monomeric moiety, a ubiquitin activating enzyme (E1), a ubiquitin-conjugating enzyme (E2) and optionally a ubiquitin-ligating enzyme (E3);
wherein the or each monomeric moiety comprises a ubiquitin having G-76 available for formation of an amide bond at its C-terminus; and
(ii) thereby forming the multimeric conjugate comprising at least three conjugated monomeric moieties, such that:
a first monomeric moiety is conjugated to a second monomeric moiety via an amide bond from G76 of the first monomeric moiety's ubiquitin to one of K6, K11, K27, K29, K33, K48, or K63 of the second monomeric moiety's ubiquitin; and
the second monomeric moiety is conjugated to a third monomeric moiety via an amide bond from G76 of the second monomeric moiety's ubiquitin to one of K6, K11, K27, K29, K33, K48, or K63 of the third monomeric moiety's ubiquitin.

56. The method of claim 55, wherein the solution comprises a monomeric moiety, a ubiquitin activating enzyme (E1), a ubiquitin-conjugating enzyme (E2) and a ubiquitin-ligating enzyme (E3).

57. The method of claim 55, wherein the first monomeric moiety is conjugated to the second monomeric moiety via an amide bond from G76 of the first monomeric moiety's ubiquitin to K48 or K63 second monomeric moiety's ubiquitin; and
the second monomeric moiety is conjugated to the third monomeric moiety via an amide bond from G76 of the second monomeric moiety's ubiquitin to K48 or K63 third monomeric moiety's ubiquitin.

58. The method of claim 55, wherein the E2 and E3 enzymes are Mms2 and Ubc13, or Ubc13 and Uev1A, or UbcH7 and Gp78, or UbcH7 and NleL.

59. The method of claim 55, wherein the or each monomeric moiety comprises a fusion protein with the ubiquitin at its C-terminus.

60. The method of claim 59, wherein the or each fusion protein comprises a biologically and/or pharmaceutically active polypeptide or peptide and the ubiquitin at its C-terminus.

61. The method of claim 59, wherein the or each fusion protein comprises an MEW.

62. The method of claim 55, further comprising:
(iii) optionally isolating the multimeric conjugate formed in step (ii);
(iv) providing a solution comprising the (optionally isolated) multimeric conjugate formed in step (ii), a distal monomeric moiety and/or a proximal monomeric moiety, a ubiquitin activating enzyme (E1), a ubiquitin-conjugating enzyme (E2) and optionally a ubiquitin-ligating enzyme (E3);
wherein the distal monomeric moiety, when present, comprises a distal ubiquitin at its C-terminus, the distal ubiquitin optionally comprising at least one of the following mutations: K6X, K11X, K27X, K29X, K33X, K48X, or K63X, where X is selected from R, A or C;
wherein the proximal monomeric moiety, when present, comprises a polypeptide comprising a proximal ubiquitin at its C-terminus, or a proximal ubiquitin at it N-terminus, said ubiquitin comprising a blocked C-terminus; and
(v) thereby forming a second multimeric conjugate such that the distal moiety, when present, is conjugated to the first monomeric moiety via an amide bond from G76 of the distal ubiquitin to one of K6, K11, K27, K29, K33, K48, or K63 of the first monomeric moiety's ubiquitin; and such that the proximal moiety, when present, is conjugated to the most proximal moiety of the multimeric conjugate formed in step (ii) via an amide bond from G76 of the ubiquitin of the most proximal moiety of the multimeric conjugate formed in step (ii) to one of K6, K11, K27, K29, K33, K48, or K63 of the proximal ubiquitin.

63. The method of claim 62, wherein the distal ubiquitin is substituted with a probe and/or wherein the proximal ubiquitin is substituted with a probe.

64. The method of claim 63, wherein the or each probe is a label, optionally a fluorophore.

65. A conjugate obtainable by or obtained by a method of claim 34.

66. A formulation comprising the conjugate of claim 1, and optionally a pharmaceutically acceptable carrier.

67. A conjugate of claim 1, for use as a medicament.

68. A conjugate of claim 1 for use in the treatment of cancer, an autoimmune disease, Alzheimer's disease, or a genetic disorder.

69. A method of deactivating a conjugate of claim 1, comprising contacting the conjugate with a deubiquitinating enzyme, such that a linkage between at least two of the moieties of the conjugate is cleaved.


SEQUENCE LISTING (6 sequences)

SEQ ID NO: 1 - protein, 76 aa, Homo sapiens [sequence omitted]

SEQ ID NO: 2 - protein, 213 aa, Artificial Sequence
  Ubc7-gp78RING fusion protein based on Ubc7 of AMFR_HUMAN  and gp78RING domain of UB2G2_HUMAN
EARFAVATPEELAVNNDDCAICWDSMQAARKLPCGHLFHNSCLRSWLEQDTSCPTCRMSLNIADNNRVREEGTGSHMNEENFFEWEALIMGPEDTCFEFGVFPAILSFPLDYPLSPPKMRFTCEMFHPNIYPDGRVCISILHAPGDDPMGYESSAERWSPVQSVEKILLSVVSMLAEPNDESGANVDASKMWRDDREQFYKIAKQIVQKSLGL

SEQ ID NO: 3 - protein, 76 aa, Homo sapiens
  misc_feature (6)..(6): X is R, A or C
  misc_feature (11)..(11): X is R, A or C
  misc_feature (27)..(27): X is R, A or C
  misc_feature (29)..(29): X is R, A or C
  misc_feature (33)..(33): X is R, A or C
  misc_feature (48)..(48): X is R, A or C
  misc_feature (63)..(63): X is R, A or C
  misc_feature (76)..(76): G may be deleted
  SITE (76)..(76): His-tag
MQIFVXTLTGXTITLEVEPSDTIENVXAXIQDXEGIPPDQQRLIFAGXQLEDGRTLSDYNIQXESTLHLVLRLRGG

SEQ ID NO: 4 - protein, 8 aa, Artificial Sequence
  Peptide SSP, amino acids 258-265 of OVAL_CHICK
SIINFEKL

SEQ ID NO: 5 - protein, 24 aa, Artificial Sequence
  Peptide SLP, amino acids 248-265 of OVAL_CHICK and A5K
DEVSGLEQLESIINFEKLAAAAAK

SEQ ID NO: 6 - protein, 366 aa, Artificial Sequence
  Fusion protein based on MHC-I H-2Kb and UBB_HUMAN
MMGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPGSGGSGGSAGGMQIFVKTLTGKTITLEVEPSDTIENVKAKIQDKEGIPPDQQRLIFAGKQLEDGRTLSDYNIQKESTLHLVLRLRGG